(19) United States
(12) Patent Application Publication
(10) Pub. No.: US 2010/0298370 A1
(43) Pub. Date: Nov. 25, 2010

(54) BENZOTHIAZOLYL THIENOPYRIDINE DERIVATIVES AND USES THEREOF

(76) Inventors: Paul Gregor, Rehovot (IL); Regina Zhuk, Ness-Ziona (IL); Nicolas Harris, Rehovot (IL); Juraj Koppel, Kosice (IL)

Correspondence Address: BROWDY AND NEIMARK, P.L.L.C.;624 NINTH STREET, NW, SUITE 300, WASHINGTON, DC, 20001-5303, US

(21) Appl. No.: 12/377,536

(22) PCT Filed: Aug. 14, 2007
(86) PCT No.: PCT/IL07/01016

§ 371 Date: Feb. 13, 2009

(30) Foreign Application Priority Data
Mar. 20, 2006 (GB) 0605581.8

(51) Int. Cl. A61K 31/4365; C07D 495/04; A61P 37/06; A61P 31/00; A61P 11/06; A61P 9/10; A61P 29/00; A61P 19/02; A61P 17/06; A61P 35/00

(52) U.S. Cl. 514/301; 546/114

(57) ABSTRACT

Novel benzothiazolyl thienopyridine compounds are provided and pharmaceutical compositions comprising benzothiazolyl thienopyridine compounds. The benzothiazolyl thienopyridine compounds are capable of inhibiting the interactions between vascular endothelial growth factor (VEGF) and heparan sulfate glycosaminoglycans (HS-GAGs), and are useful for prevention or treatment of diseases and disorders such as inflammation, autoimmune diseases and cancer.

### FIELD OF INVENTION

The present invention relates to novel benzothiazolyl thienopyridine compounds and to pharmaceutical compositions comprising benzothiazolyl thienopyridine compounds capable of inhibiting the interactions between vascular endothelial growth factor (VEGF) and heparan sulfate glycosaminoglycans (HS-GAGs). The benzothiazolyl thienopyridine compounds are useful for the prevention or treatment of diseases and disorders such as inflammation, autoimmune diseases and cancer.

### BACKGROUND OF THE INVENTION

The inflammatory response is mediated primarily by leukocytes, neutrophils and lymphocytes, which circulate in the blood and reversibly interact with the vascular endothelium. In response to inflammatory stimuli, the leukocytes adhere tightly to the vascular endothelium, migrate (extravasate) through the vessel wall, and subsequently move along a chemotactic gradient toward the inflammatory stimulus.

Glycosaminoglycans (also referred to herein as “GAG” or “GAGs”) are naturally-occurring carbohydrate-based molecules implicated in the regulation of a number of cellular processes, including blood coagulation, angiogenesis, tumor growth, and smooth muscle cell proliferation, most likely by interaction with effector molecules. GAGs are often, but not always, found covalently bound to protein cores in structures called proteoglycans. Proteoglycan structures are abundant on cell surfaces and are associated with the extracellular matrix around cells. GAGs consist of repeating disaccharide units. For example, heparan sulfate glycosaminoglycans (also referred to herein as “HS-GAGs”) consist of repeating disaccharide units of D-glucuronic acid and N-acetyl- or N-sulfo-D-glucosamine. The high molecular diversity of HS-GAGs is due to their unique sulfation pattern (Sasisekharan and Venkataraman, 2000). One of the most thoroughly studied HS-GAGs is the widely used anticoagulant heparin. Heparin is a highly sulfated form of heparan sulfate found in mast cells. Many important regulatory proteins including cytokines, growth factors, enzymes, and cell adhesion molecules bind tightly to heparin. Although interactions of proteins with GAGs such as heparin and heparan sulfate are of great biological importance, the structural requirements for protein-GAG binding have not been well characterized. Ionic interactions are important in promoting protein-GAG binding and the spacing of the charged residues may determine protein-GAG affinity and specificity.

The conventional concept is that cytokines in solution act as diffusible factors. However, recent progress in cytokine research suggests that many cytokines can function in a non-diffusible fashion when immobilized on either the cell surface or the extracellular matrix (ECM) by binding to heparan sulfate proteoglycan (HS-PG). The number of cytokines that contain heparin-binding domains and can thus potentially be immobilized on HS-GAGs is large and includes for instance MIP-1, MCP-3, RANTES, IL-8, VEGF, IL-6, IL-3, IL-7 and IFN-gamma.

Chemokines are small chemoattractant cytokines that control a wide variety of biological and pathological processes, ranging from immunosurveillance to inflammation, and from viral infection to cancer. Genetic and pharmacological studies have shown that chemokines are responsible for the excessive recruitment of leucocytes to inflammatory sites and damaged tissue. Chemokines have an in vivo requirement to bind to GAGs in order to mediate directional cell migration. Prevention of the GAG interaction has been shown to be a viable therapeutic strategy (Johnson et al., 2004). The chemokine system offers many potential entry points for innovative anti-inflammatory therapies for autoimmune diseases, such as multiple sclerosis, rheumatoid arthritis and allergic contact dermatitis. The ability of certain cytokines to bind to and be immobilized by HS-GAGs accentuates their action by: 1) promoting the accumulation of cytokines at high concentrations in the appropriate location to encounter their target cells; 2) activating cytokines by inducing conformational changes in the bound cytokine; 3) promoting conformation-dependent association or polymerization of cytokines and their receptors and facilitating the assembly of the appropriate molecular complex to initiate signal transduction; and 4) protecting cytokines from both chemical and physiologic degradation (Tanaka et al., 1998).

VEGF acts as a pro-inflammatory cytokine by increasing endothelial permeability and inducing adhesion molecules that bind leukocytes to endothelial cells and via its ability to act as a monocyte chemoattractant (Kim et al., 2001; Melder et al., 1996). A single VEGF gene gives a few isoforms through alternative splicing. Isoforms VEGF165, VEGF189, and VEGF206 bind heparin with increasingly greater affinity, whereas VEGF121 does not bind heparin. The different VEGF isoforms share a common amino-terminal receptor-binding domain of 115 residues, and the heparin-binding forms share the same carboxy-terminal 50 residues, which constitute a heparin-binding domain which mediates interaction with HS-GAGs (Wayne et al., 1998). The strong interaction between VEGF and Heparin/HS-GAGs is stemming from highly sulfated GAG sequences and a VEGF interaction with two S-domains on the same Heparin/HS-GAG chain (Christopher et al., 2006).

VEGF plays a key role in most aspects of vascular development and functions such as angiogenesis, vasculogenesis, lymphangiogenesis, cancer, ischemia and inflammation (Christopher et al., 2006). VEGF plays an important role in the induction of several inflammatory diseases such as multiple sclerosis (MS) (Su et al., 2006); rheumatoid arthritis (RA) (Kasama et al., 2000; Maruotti et al., 2006); atherosclerosis (Rohit et al., 2005); inflammatory bowel disease (IBD) (Kanazawa et al., 2001; Di Sabatino et al., 2004); systemic lupus erythematosus (SLE) (Navarro et al., 2002) and ischemia/reperfusion injury (IRI) (Tsuchihashi et al., 2006).

Japanese Patent Application JP 2001151779 discloses 4,5,6,7-tetrahydrothieno[2,3-c]pyridines as anti-TNF-α agents (see also Fujita M. et al., 2002a). Japanese Patent Application JP 2001151780 discloses 4,5,6,7-tetrahydrothieno[2,3-c]pyridines as inhibitors of TNF-α synthesis (see also Fujita M. et al., 2002b). The 4,5,6,7-tetrahydrothieno[2,3-c]pyridines have different substituents in the position 3 of the ring: arylcarbonyl in JP 2001151779 and carboxy or alkoxycarbonyl in JP 2001151780. As will be described hereinafter, these compounds are different from those of the present invention as they do not contain the benzothiazole ring attached directly to the backbone (and not through a carbonyl) at the position 3. Moreover, in JP 2001151780, R3 (corresponding to R1 in the present invention) is defined as a lower alkanoyl or a cycloalkylcarbonyl as an essential feature of the invention.

WO 2004/069149 of the same applicant discloses thieno[2,3-c]pyridine derivatives that inhibit GAG-L-selectin interaction, comprising of thieno[2,3-c]pyridine scaffold, a benzoyl amide residue in the middle of the molecule and a sulfonamide residue as a substituent on the phenyl ring. The compounds of the present invention differ from those disclosed in WO 2004/069149, since they do not possess a sulfonamide substituent.

As far as known by Applicant, the background art has not taught or suggested synthesis of the novel benzothiazolyl thienopyridine compounds of the general formula I of the present invention, and that benzothiazolyl thienopyridine compounds of the general formulas I and II described herein in the present application have any biological or pharmaceutical activity.

### SUMMARY OF THE INVENTION

It is an object of the present invention to provide novel benzothiazolyl thieno[2,3-c]pyridine compounds that are inhibitors of HS-GAG/VEGF interaction and may be useful, for instance, for the treatment or prevention of inflammatory and autoimmune diseases and disorders.

According to one aspect, the present invention provides novel compounds of the general formula I:

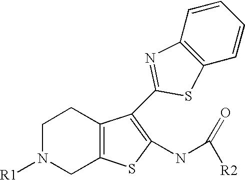

wherein:

R1 is optionally substituted hydrocarbyl, alkylcarbonyl or alkoxycarbonyl; and

R2 is [(4-methylthio)phenyl]methyl, (3-methoxyphenoxy)methyl, benzo[b]fur-2-yl or 1,3-dihydro-1,3-dioxo-2H-isoindol-2-yl-ethyl.

According to one embodiment, the compounds of the general formula I inhibit the interactions of HS-GAGs with VEGF. According to another embodiment, the compounds of general formula I exhibit anti-inflammatory activity in vivo and thus may be useful for prevention or treatment of inflammatory and autoimmune diseases.

According to preferred embodiments, the novel compounds of the general formula I are the compounds:
N-[3-(2-benzothiazolyl)-4,5,6,7-tetrahydro-6-methylthieno[2,3-c]pyridin-2-yl]-2-(4-methylthiophenyl)acetamide (Compound 3);
N-[3-(2-benzothiazolyl)-4,5,6,7-tetrahydro-6-methylthieno[2,3-c]pyridin-2-yl]-2-(3-methoxyphenoxy)acetamide (Compound 4);
N-[3-(2-benzothiazolyl)-4,5,6,7-tetrahydro-6-methylthieno[2,3-c]pyridin-2-yl]-benzo[b]furan-2-carboxamide (Compound 5);
N-[3-(2-benzothiazolyl)-4,5,6,7-tetrahydro-6-methylthieno[2,3-c]pyridin-2-yl]-3-(1,3-dihydro-1,3-dioxo-2H-isoindol-2-yl)propanamide (Compound 6).

Another object of the present invention is to provide pharmaceutical compositions comprising small organic compounds for medical and diagnostic use, wherein the small organic compounds are inhibitors of the interactions between VEGF and HS-GAGs, and may be useful in the prevention or treatment of diseases and disorders such as inflammation, autoimmune diseases and cancer.

According to a preferred embodiment, the present invention provides a pharmaceutical composition comprising a pharmaceutically acceptable diluent or carrier and a compound of the general formula II:

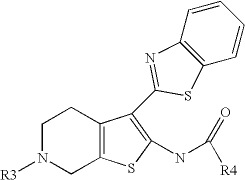

wherein:

R3 is optionally substituted hydrocarbyl, alkylcarbonyl or alkoxycarbonyl;

R4 is optionally substituted hydrocarbyl, aryloxyalkyl, arylthioalkyl, arylsulfonylalkyl or heterocyclyl;

and pharmaceutically acceptable salts thereof.

The compounds of general formula II inhibit the interactions of HS-GAGs with VEGF and may thus be useful in the treatment or prevention of diseases or disorders mediated by VEGF-HS-GAGs interaction including acute and chronic inflammatory and autoimmune disorders dr diseases exemplified by, but not restricted to, sepsis, ischemia-reperfusion injury, cardiomyopathic disease, infectious meningitis, encephalitis, acute respiratory distress syndrome, organ/tissue transplant rejection (such as skin, kidney, heart, lung, liver, bone marrow, cornea, pancreas, small bowel), lupus, atherosclerosis, septic shock, post-ischemic leukocyte-mediated tissue damage, frost-bite injury or shock, acute leukocyte-mediated lung injury, acute pancreatitis, nephritis, asthma, traumatic shock, stroke, Crohn's disease, traumatic brain injury, acute and chronic inflammation, atopic dermatitis, uveitis, colitis, inflammatory bowel disease, rheumatoid arthritis, retinitis, psoriasis and multiple sclerosis.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a graph showing the anti-inflammatory activity of Compounds 1-6 administered to mice intraperitoneally (i.p.) at 20 mg/kg, 1 hour before induction of paw edema (10-12 mice per group). The swelling of paws was measured 4 hours after induction, compared to controls and expressed as difference in paw thickness in mm (y-axis). The results were statistically evaluated by Student's t-test and statistical significance is indicated by one, two or three stars (*p>0.05; ** p>0.01; *** p>0.001).

FIG. 2 is a graph showing the anti-inflammatory activity of Compounds 1, 2, 4, 5, 6 administered to mice orally at 50 mg/kg in a model of delayed-type hypersensitivity (DTH) (11-13 mice per group). The results for dexamethasone (10 mg/kg i.p.), a steroid positive control, are also shown. The y-axis represents difference in ear thickness in mm compared to control. The results were statistically evaluated by Student's t-test and statistical significance is indicated by one, two or three stars (*p>0.05; ** p>0.01; *** p>0.001).

### DETAILED DESCRIPTION OF THE INVENTION

The present invention provides, in one aspect, novel compounds of the general formula I:

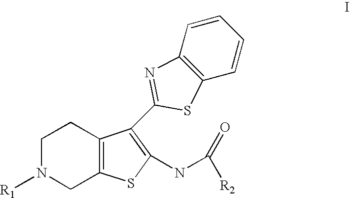

wherein

R₁ is optionally substituted hydrocarbyl, alkylcarbonyl or alkoxycarbonyl;

R₂ is selected from [(4-methylthio)phenyl]methyl, (3-methoxyphenoxy)methyl, benzo[b]fur-2-yl and 1,3-dihydro-1,3-dioxo-2H-isoindol-2-yl-ethyl.

In preferred embodiments, the invention provides the novel compounds herein designated Compounds 3, 4, 5 and 6.

The present invention further provides the synthesis of the compounds of general formula: a general synthetic route for synthesis of such compounds is described in Example 1 and synthesis of the four novel compounds 3-6 is described in Examples 2 to 5 hereinafter.

According to another aspect, the present invention provides a pharmaceutical composition comprising a pharmaceutically acceptable diluent or carrier and at least one compound of the general formula II:

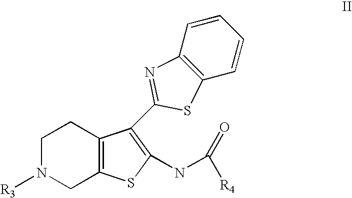

wherein:

R₃ is optionally substituted hydrocarbyl, alkylcarbonyl or alkoxycarbonyl;

R₄ is optionally substituted hydrocarbyl, aryloxyalkyl, arylthioalkyl, arylsulfonylalkyl or heterocyclyl;

and pharmaceutically acceptable salts thereof.

As used herein, the term “hydrocarbyl” refers to a radical derived from a hydrocarbon, which consists only of carbon and hydrogen atoms, and no other elements. The hydrocarbyl is preferably a (C₁-C₂₀)hydrocarbyl, which may be acyclic or cyclic, saturated or unsaturated and includes alkyl, alkenyl, alkynyl, cycloalkyl, cycloalkenyl, aralkyl and aryl.

The term “alkyl” as defined herein, alone or in combination, typically refers to a straight or branched alkyl radical, preferably having 1-6 carbon atoms, i.e., (C₁-C₆)alkyl, and includes, for example, methyl, ethyl, n-propyl, isopropyl, n-butyl, isobutyl, tert-butyl, n-pentyl, isopentyl, 2,2-dimethylpropyl, and n-hexyl.

The term “cycloalkyl”, alone or in combination, means a saturated or partially saturated monocyclic, bicyclic or tricyclic alkyl radical wherein each cyclic moiety contains from about 3 to about 8 carbon atoms, more preferably from about 3 to about 6 carbon atoms. Examples of such cycloalkyl radicals include cyclopropyl, cyclobutyl, cyclopentyl, cyclohexyl and the like.

The term “aryl”, alone or in combination, refers to an aromatic carbocyclic group preferably of 6 to 20, more preferably 6 to 10 carbon atoms, i.e., (C₆-C₂₀) or (C₆-C₁₀)aryl, respectively, such as phenyl and naphthyl.

The term “heterocyclyl”, alone or in combination, refers to a radical derived from a mono- or poly-cyclic ring containing one to three heteroatoms selected from the group consisting of N, O and S, with or without unsaturation or aromatic character. The term “heteroaryl” refers to such a mono- or poly-cyclic ring having aromatic character. Non-limiting examples of non-aromatic heterocyclyl include dihydrofuryl, tetrahydrofuryl, dihydrothienyl, pyrrolydinyl, pyrrolynyl, dihydropyridyl, piperidinyl, piperazinyl, morpholino, 1,3-dioxanyl, 1,3-dioxo-isoindolyl and the like. A polycyclic ring may have the rings fused, as in quinoline or benzofuran, or unfused as in 4-phenylpyridine. Non-limiting examples of heteroaryl include pyrrolyl, furyl, thienyl, pyrazolyl, imidazolyl, oxazolyl, isoxazolyl thiazolyl, isothiazolyl, pyridyl, 1,3-benzodioxinyl, pyrazinyl, pyrimidinyl, 1,3,4-triazinyl, 1,2,3-triazinyl, 1,3,5-triazinyl, thiazinyl, quinolinyl, isoquinolinyl, benzofuryl, isobenzofuryl, indolyl, imidazo[1,2-a]pyridyl, pyrido[1,2-a]pyrimidinyl, benzimidazolyl, benzthiazolyl, benzothienyl, benzoxazolyl, 4-oxo-benzopyranyl, 1,3-benzodioxolyl, 3-oxo-3H-naphto[2,1-b]pyranyl, 1,4-benzo-dioxinyl and the like. It is to be understood that when a polycyclic heteroaromatic ring is substituted, the substitutions may be in any of the carbocyclic and/or heterocyclic rings.

Any alkyl, cycloalkyl, aryl, heteroaryl or heterocyclyl radical may be substituted by one or more radicals including, but not limited to, halogen, hydroxy, (C₁-C₁₀)alkyl, (C₂-C₁₀)alkenyl, (C₂-C₁₀)alkynyl, (C₇-C₁₂)aralkyl, (C₆-C₁₀)aryl, (C₇-C₁₂)alkaryl, (C₁-C₁₀)alkoxy, (C₆-C₁₀)aryloxy, (C₁-C₁₀)alkylthio, (C₆-C₁₀)arylthio, (C₆-C₁₀)arylamino, (C₃-C₁₀)cycloalkyl, (C₃-C₁₀)cycloalkenyl, amino, (C₁-C₁₀)alkylamino, di(C₁-C₁₀)-alkylamino, (C₂-C₁₂)alkoxyalkyl, (C₂-C₁₂)alkylthio-alkyl, (C₁-C₁₀)alkylsulfinyl, (C₁-C₁₀)alkylsulfonyl, (C₆-C₁₀)arylsulfonyl, hydroxy-(C₁-C₁₀)alkyl, (C₆-C₁₀)aryloxy(C₁-C₁₀)alkyl, (C₁-C₁₀)alkoxycarbonyl, (C₆-C₁₀)aryl-oxycarbonyl, (C₂-C₁₁) alkanoyl, (C₇-C₁₁)aroyl, fluoro(C₁-C₁₀)alkyl, oxo, nitro, nitro-(C₁-C₁₀)alkyl, cyano, cyano(C₁-C₁₀)alkyl, aminocarbonyl, (C₁-C₁₀)alkyl-aminocarbonyl, di(C₁-C₁₀)-alkylaminocarbonyl, aminocarbonyl(C₁-C₁₀)alkyl, aminocarbonyl(C₆-C₁₀)aryl, aminosulfonyl, (C₁-C₁₀)alkylaminosulfonyl, di(C₁-C₁₀)-alkylaminosulfonyl, amidino, carboxy, sulfo, heterocyclyl, and —(CH₂)ₘ—Z—(C₁-C₁₀)alkyl, where m is 1 to 8 and Z is oxygen or sulfur.

As used herein, the term “(C₁-C₁₀)alkoxy” refers to the group (C₁-C₁₀)alkyl-O—, wherein (C₁-C₁₀)alkyl has the significance given above. Examples of alkoxy are methoxy, ethoxy, butoxy, hexoxy, and the like.

The term “(C₆-C₁₀)aryloxy” refers to the group (C₆-C₁₀)aryl-O—, wherein (C₆-C₁₀)aryl is as defined above. Examples of aryloxy are phenoxy and naphthyloxy.

The terms “(C₁-C₁₀)alkylthio” and “(C₆-C₁₀)arylthio”, as used herein refer to the groups (C₁-C₁₀)alkyl-S— and (C₆-C₁₀)aryl-S—, respectively, wherein the terms alkyl and aryl are as defined above. Examples are methylthio and phenylthio.

The terms “alkylsulfonyl” and “arylsulfonyl” refer to the radicals alkyl-SO₂— and aryl-SO₂—, respectively, preferably (C₁-C₁₀)alkyl-SO₂— and (C₆-C₂₀)aryl-SO₂—, more preferably (C₁-C₆)alkyl-SO₂— and (C₆-C₁₀)aryl-SO₂—, respectively.

The terms “alkylcarbonyl” or “alkanoyl” refer to an acyl radical of the formula alkyl-CO—, preferably (C₁-C₆)alkyl-CO—, derived from an alkanecarboxylic acid.

The term “alkyl” has the significance given above. Examples of alkylcarbonyl include acetyl, propionyl, butyryl, valeryl, 4-methylvaleryl, and the like.

The term “alkoxycarbonyl” refers to a radical of the formula alkyl-O—CO—, preferably (C₁-C₁₀)alkoxycarbonyl, more preferably (C₁-C₆)alkoxycarbonyl, in which the term alkyl has the significance given above. Examples of alkoxycarbonyl radical are methoxycarbonyl, ethoxycarbonyl and the like.

The term “aryloxyalkyl” refers to a radical of the formula aryl-O-alkyl, preferably (C₆-C₁₀)aryloxy(C₁-C₁₀)alkyl, more preferably (C₆-C₁₀)aryloxy(C₁-C₆)alkyl, in which the terms “aryl” and “alkyl” have the significance given above. An example of an aryloxyalkyl is phenoxymethyl.

The term “arylthioalkyl” refers to a radical of the formula aryl-5-alkyl, preferably (C₆-C₂₀)arylthio(C₁-C₁₀)alkyl, more preferably (C₆-C₁₀)arylthio(C₁-C₆)alkyl, in which the terms “aryl” and “alkyl” have the significance given above. Examples of arylthioalkyls are phenylthiomethyl, phenylthiobutyl and the like.

The term “arylsulfonylalkyl” refers to a radical of the formula aryl-SO₂-alkyl, preferably (C₆-C₂₀)arylsulfonyl(C₁-C₁₀)alkyl, more preferably (C₆-C₁₀)arylsulfonyl-(C₁-C₆)alkyl, in which the terms “aryl” and “alkyl” have the significance given above. An example of an arylsulfonylalkyl is phenylsulfonylmethyl.

The term “amino” as used herein includes a primary, secondary or tertiary amino group containing substituents selected from alkyl, aryl, aralkyl, cycloalkyl, cycloalkylalkyl radicals and the like, all as defined herein.

The term “halogen” refers to fluoro, chloro, bromo or iodo.

It is to be understood that the term “substituted”, as used herein, means that any one or more hydrogen on the designated atom is replaced with a selection from the indicated groups, provided that the designated atom's normal valency is not exceeded, and that the substitution results in a stable compound. Combinations of substituents are permissible only if such combinations result in stable compounds. By “stable compound” or “stable structure” it is meant herein a compound that is sufficiently robust to survive isolation to a useful degree of purity from a reaction mixture, and formulation into an efficacious therapeutic agent.

As contemplated herein, the present invention further encompasses isomers, pharmaceutically acceptable salts and hydrates of the compounds defined by the present invention.

The term “isomer” includes, but is not limited to, optical isomers, structural isomers, conformational isomers, and the like. Thus, the present invention encompasses various optical isomers of the compounds of formula I or II. It will be appreciated by those skilled in the art that the compounds of the present invention contain at least one chiral center. Accordingly, these compounds exist in, and are isolated in, optically active or racemic forms. Unless otherwise indicated, all chiral, diastereomeric and racemic forms of the compounds described in the present invention are encompassed by the present invention. The compounds may also have asymmetric centers. Many geometric isomers of olefins, C— and N— double bonds and the like can also be present in the compounds described herein, and all such stable isomers are contemplated in the present invention. It will be appreciated that compounds of the present invention that contain asymmetrically substituted carbon atoms may be isolated in optically active or racemic forms. It is well known in the art how to prepare optically active forms, for example, by resolution of the racemic form by recrystallization techniques, by synthesis from optically active starting materials, by chiral synthesis, or by chromatographic separation using a chiral stationary phase. All chiral, diastereomeric, racemic forms and all geometric isomeric forms of a structure are intended, unless the specific stereochemistry or isomer form is specifically indicated.

Some compounds may also exhibit polymorphism. It is to be understood that the present invention encompasses any racemic, optically active, polymorphic, or stereroisomeric form, or mixtures thereof. In one embodiment, the compounds are the pure (R)-isomers. In another embodiment, the compounds are the pure (S)-isomers. In another embodiment, the compounds are a mixture of the (R) and the (S) isomers. In another embodiment, the compounds are a racemic mixture comprising an equal amount of the (R) and the (S) isomers.

In addition, this invention further includes hydrates of the compounds described herein. The term “hydrate” includes, but is not limited to hemihydrate, monohydrate, dihydrate, trihydrate, and the like.

The compounds of the present invention can also be in the form of prodrugs. Prodrugs are considered to be any covalently bonded carriers that release the active parent drug of general formula I or II in vivo, when such prodrug is administered to a mammalian subject. Prodrugs of the compounds of Formula I or II are prepared by modifying functional groups present in the compounds in such a way that the modifications are cleaved, either in routine manipulation or in vivo, to provide the parent compound of formula I or II. Prodrugs include compounds of formula I and/or II wherein hydroxyl, amino, sulfhydryl or carboxyl groups are bonded to any group that, when administered to a mammalian subject, are cleaved to form a free hydroxyl, amino, sulfhydryl or carboxyl group, respectively. Examples of prodrugs include, but are not limited to, acetate, formate, and benzoate derivatives of hydroxy and amino functional groups in the compounds of Formula I or II, and the like.

The present invention encompasses also pharmaceutically acceptable salts of the compounds of the present invention. Pharmaceutically acceptable salts can be prepared by reaction with inorganic bases, for example, sodium hydroxide or inorganic/organic acids such as hydrochloric acid, citric acids and the like.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic, manganous, potassium, sodium, or zinc salts and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It is to be understood that, as used herein, references to the compounds according to formula I and II of the present invention are meant to also include the pharmaceutically acceptable salts thereof.

According to one preferred embodiment of the invention, the pharmaceutical composition comprises at least one compound of the general formula II, wherein:

R₃ is selected from the group consisting of (C₁-C₆)alkyl, (C₆-C₁₀)aryl, (C₆-C₁₀)aryl(C₁-C₆)alkyl, (C₁-C₆)alkyl carbonyl and (C₁-C₆)alkoxycarbonyl; and

R₄ is selected from the group consisting of (C₁-C₆)alkyl, (C₃-C₈)cycloalkyl, (C₆-C₂₀)aryl, (C₆-C₂₀)aryl(C₁-C₆)alkyl, (C₆-C₂₀)aryloxy(C₁-C₆)alkyl, (C₆-C₂₀)arylthio(C₁-C₆)alkyl, (C₆-C₂₀)arylsulfonyl(C₁-C₆)alkyl, (C₄-C₁₂)heteroaryl or (C₆-C₁₀)bicyclic heterocyclyl;

wherein each (C₁-C₆)alkyl, (C₆-C₁₀) or (C₆-C₂₀)aryl, either alone or as part of an aralkyl, alkylcarbonyl, alkoxycarbonyl, aryloxyalkyl, arylthioalkyl or arylsulfonylalkyl moiety, and any heteroaryl or heterocyclyl may be optionally substituted by one to three radicals selected from the group consisting of halogen, cyano, amino, hydroxy, oxo, nitro, (C₁-C₆)alkyl, (C₁-C₆)haloalkyl, (C₃-C₈)cycloalkyl, (C₁-C₆)alkoxy, (C₁-C₆)alkylcarbonyl, (C₁-C₆)alkoxycarbonyl, (C₁-C₆)alkylsulfonyl, (C₁-C₆)alkylthio and heterocyclyl.

According to more preferred embodiments, the pharmaceutical composition of the invention comprises at least one compound of the general formula II, wherein:

R₃ is selected from the group consisting of a straight or branched (C₁-C₃) alkyl, benzyl, acetyl and ethoxycarbonyl; and

R4 is selected from the group consisting of:
(i) straight or branched (C₁-C₄)alkyl, unsubstituted or substituted by (C₃-C₆)cycloalkyl, 2,5-dioxo-pyrrolidin-1-yl or 1,3-dioxo-isoindol-2-yl;
(ii) (C₃-C₆)cycloalkyl;
(iii) phenyl, unsubstituted or substituted by one to three radicals selected from halogen, cyano, dimethylamino, trifluoromethyl, (C₁-C₃)alkoxy, (C₁-C₃)alkylcarbonyl, (C₁-C₃)alkoxycarbonyl or 2,5-dioxo-pyrrolidin-2-yl;
(iv) 1-naphthyl or 2-naphthyl, substituted by (C₁-C₃)alkoxy;
(v) phenyl(C₁-C₃)alkyl, unsubstituted or substituted by methylthio on the phenyl moiety;
(vi) phenoxy(C₁-C₃)alkyl, unsubstituted or substituted on the phenyl moiety by one to three radicals selected from the group consisting of halogen, alkoxy or acetyl;
(vii) phenylthio(C₁-C₃)alkyl, unsubstituted or substituted on the phenyl moiety by one to three (C₁-C₃)alkyl radicals;
(viii) phenylsulfonyl(C₁-C₃)alkyl, unsubstituted or substituted on the phenyl moiety by one to three (C₁-C₃)alkyl radicals;
(ix) heteroaryl selected from the group consisting of: furyl or thienyl, unsubstituted or substituted by one or two halogen atoms; benzofuryl; benzothienyl substituted by a nitro radical; and benzthiazolyl; and
(x) heterocyclyl selected from the group consisting of 1,3-benzodioxolyl; 4-oxo-benzopyranyl, 3-oxo-3H-naphto[2,1-b]pyranyl and 1,4-benzodioxinyl.

According to still more preferred embodiments, the pharmaceutical composition of the invention comprises, as active agent, a compound of formula I or II selected from the group consisting of Compounds 1-40:
N-[3-(2-benzothiazolyl)-4,5,6,7-tetrahydro-6-ethylthieno[2,3-c]pyridin-2-yl]-4-cyanobenzamide (Compound 1);
N-[3-(2-benzothiazolyl)-4,5,6,7-tetrahydro-6-(1-methylethyl)thieno[2,3-c]pyridin-2-yl]-4-(dimethylamino)benzamide (Compound 2);
N-[3-(2-benzothiazolyl)-4,5,6,7-tetrahydro-6-methylthieno[2,3-c]pyridin-2-yl]-2-(4-methylthiophenyl)acetamide (Compound 3);
N-[3-(2-benzothiazolyl)-4,5,6,7-tetrahydro-6-methylthieno[2,3-c]pyridin-2-yl]-2-(3-methoxyphenoxy)acetamide (Compound 4);
N-[3-(2-benzothiazolyl)-4,5,6,7-tetrahydro-6-methylthieno[2,3-c]pyridin-2-yl]-benzo[b]furan-2-carboxamide (Compound 5);
N-[3-(2-benzothiazolyl)-4,5,6,7-tetrahydro-6-methylthieno[2,3-c]pyridin-2-yl]-3-(1,3-dihydro-1,3-dioxo-2H-isoindole-2)propanamide (Compound 6);
N-[3-(2-benzothiazolyl)-4,5,6,7-tetrahydro-6-methylthieno[2,3-c]pyridin-2-yl]-2-thiophenecarboxamide (Compound 7);
N-[3-(2-benzothiazolyl)-4,5,6,7-tetrahydro-6-phenylmethylthieno[2,3-c]pyridin-2-yl]-4-acetylbenzamide (Compound 8);
N-[3-(2-benzothiazolyl)-4,5,6,7-tetrahydro-6-ethylthieno[2,3-c]pyridin-2-yl]-benzamide (Compound 9);
N-[3-(2-benzothiazolyl)-4,5,6,7-tetrahydro-6-ethylthieno[2,3-c]pyridin-2-yl]-(4-chloro)benzamide (Compound 10);
N-[3-(2-benzothiazolyl)-4,5,6,7-tetrahydro-6-ethylthieno[2,3-c]pyridin-2-yl]-4-methoxybenzamide (Compound 11);
N-[3-(2-benzothiazolyl)-4,5,6,7-tetrahydro-6-methylthieno[2,3-c]pyridin-2-yl]-3-[(4-fluorophenyl)thio]propanamide (Compound 12);
N-[3-(2-benzothiazolyl)-4,5,6,7-tetrahydro-6-phenylmethylthieno[2,3-c]pyridin-2-yl]-(2,4-dimethoxy)benzamide (Compound 13);
N-[3-(2-benzothiazolyl)-4,5,6,7-tetrahydro-6-ethylthieno[2,3-c]pyridin-2-yl]-(3,4,5-trimethoxy)benzamide (Compound 14);
N-[3-(2-benzothiazolyl)-4,5,6,7-tetrahydro-6-methylthieno[2,3-c]pyridin-2-yl]-3-methoxy-2-naphthalenecarboxamide (Compound 15);
N-[3-(2-benzothiazolyl)-4,5,6,7-tetrahydro-6-methylthieno[2,3-c]pyridin-2-yl]-3-trifluoromethylbenzamide (Compound 16);
N-[3-(2-benzothiazolyl)-4,5,6,7-tetrahydro-6-ethylthieno[2,3-c]pyridin-2-yl]-3-(2,5-dioxo-1-pyrrolidinyl)benzamide (Compound 17);
N-[3-(2-benzothiazolyl)-4,5,6,7-tetrahydro-6-(1-methylethyl)thieno[2,3-c]pyridin-2-yl]-3-benzenepropaneamide (Compound 18);
N-[3-(2-benzothiazolyl)-4,5,6,7-tetrahydro-6-methylthieno[2,3-c]pyridin-2-yl]-4-(1-methylethoxy)benzamide (Compound 19);
N-[3-(2-benzothiazolyl)-4,5,6,7-tetrahydro-6-(1-methylethyl)thieno[2,3-c]pyridin-2-yl]amino]carbonyl]benzoic acid methyl ester (Compound 20);
N-[3-(2-benzothiazolyl)-4,5,6,7-tetrahydro-6-phenylmethyllthieno[2,3-c]pyridin-2-yl]-2,2-dimethylpropaneamide (Compound 21);
N-[3-(2-benzothiazolyl)-4,5,6,7-tetrahydro-6-phenylmethylthieno[2,3-c]pyridin-2-yl]-3-(2,5-dichloro)thiophenecarboxamide (Compound 22);
N-[3-(2-benzothiazolyl)-4,5,6,7-tetrahydro-6-phenylmethylthieno[2,3-c]pyridin-2-yl]-2-thiophenecarboxamide (Compound 23);
N-[3-(2-benzothiazolyl)-4,5,6,7-tetrahydro-6-phenylmethylthieno[2,3-c]pyridin-2-yl]-2-(5-bromo)thiophenecarboxamide (Compound 24);
N-[3-(2-benzothiazolyl)-4,5,6,7-tetrahydro-6-methylthieno[2,3-c]pyridin-2-yl]-2-furancarboxamide (Compound 25);
N-[3-(2-benzothiazolyl)-4,5,6,7-tetrahydro-6-(1-methylethyl)thieno[2,3-c]pyridin-2-yl]-cyclohexylcarboxamide (Compound 26);
N-[3-(2-benzothiazolyl)-4,5,6,7-3-tetrahydro-6-phenylmethylthieno[2,3-c]pyridin-2-yl]-2-(4-chlorophenoxy)acetamide (Compound 27);
N-[3-(2-benzothiazolyl)-4,5,6,7-tetrahydro-6-ethylthieno[2,3-c]pyridin-2-yl]-benzo[b]thiophene-5-nitro-2-carboxamide (Compound 28);
N-[3-(2-benzothiazolyl)-4,5,6,7-tetrahydro-6-ethylthieno[2,3-c]pyridin-2-yl]-3-(phenylthio)propanamide (Compound 29);
N-[3-(2-benzothiazolyl)-4,5,6,7-tetrahydro-6-methylthieno[2,3-c]pyridin-2-yl]-1,3-dihydro-1,3-dioxo-2H-isoindole-2-acetamide (Compound 30);
N-[3-(2-benzothiazolyl)-4,5,6,7-tetrahydro-6-(methylethyl)thieno[2,3-c]pyridin-2-yl]-(2,5-dioxo-1-pyrrolidine)acetamide (Compound 31);
N-[3-(2-benzothiazolyl)-4,5,6,7-tetrahydro-6-methylthieno[2,3-c]pyridin-2-yl]-3-oxo-3H-naphtho[2,1-b]pyran-2-carboxamide (Compound 32);
N-[3-(2-benzothiazolyl)-4,5,6,7-tetrahydro-6-phenylmethylthieno[2,3-c]pyridin-2-yl]-2,3-dihydro-1,4-benzodioxin-2-carboxamide (Compound 33);
N-[3-(2-benzothiazolyl)-4,5,6,7-tetrahydro-6-methylthieno[2,3-c]pyridin-2-yl]-6-benzothiazolecarboxamide (Compound 34);
N-[3-(2-benzothiazolyl)-4,5,6,7-tetrahydro-6-ethoxycarbonylthieno[2,3-c]pyridin-2-yl]benzamide (Compound 35);
N-[3-(2-benzothiazolyl)-4,5,6,7-tetrahydro-6-acetylthieno[2,3-c]pyridin-2-yl]-3-bromobenzamide (Compound 36);
N-[3-(2-benzothiazolyl)-4,5,6,7-tetrahydro-6-acetylthieno[2,3-c]pyridin-2-yl]-4-cyanobenzamide (Compound 37);
N-[3-(2-benzothiazolyl)-4,5,6,7-tetrahydro-6-ethylthieno[2,3-c]pyridin-2-yl]-3-(phenylsulfonyl)propanamide (Compound 38);
N-[3-(2-benzothiazolyl)-4,5,6,7-tetrahydro-6-methylthieno[2,3-c]pyridin-2-yl]-(4-methylphenylsulfonyl)acetamide (Compound 39); and
N-[3-(2-benzothiazolyl)-4,5,6,7-tetrahydro-6-methylthieno[2,3-c]pyridin-2-yl]-(4-methylphenyl)thioacetamide (Compound 40).

In more preferred embodiments, the pharmaceutical composition of the invention comprises as active agent a compound of the formula I selected from the group consisting of Compound 3, 4, 5 and 6.

In accordance with the present invention and as used herein when referring to the biological activity of the compounds of general formula I or II, the following terms are defined with the following meanings, unless explicitly stated otherwise.

The term “GAG” refers to glycosaminoglycans, including heparan sulfate (HS-GAG), heparin, chondroitin sulfate, dermatan sulfate and keratan sulfate. It includes the GAG chains of proteoglycans such as heparan sulfate proteoglycan or chondroitin sulfate proteoglycan. It includes fragments of GAGs produced chemically or enzymatically as well as derivatives of GAG, which may be produced by chemical or enzymatic means as known in the art. The GAG may be free or attached to a linker, support, cell or a protein. GAGs may be crude or purified from organs, tissues or cells.

The term “HS-GAG” refers to heparan sulfate glycosaminoglycan. It includes fragments of heparan sulfate such as those that may be produced chemically, enzymatically or during purification. It includes the HS-GAG chains of proteoglycans such as heparan sulfate proteoglycans. HS-GAG may be free or attached to a linker, support, cell or protein, or otherwise chemically or enzymatically modified. HS-GAGs may be crude or purified from organs, tissues or cells. It includes heparin which is a highly sulfated species of HS-GAG.

“HS-PG” refers to heparan sulfate proteoglycans.

The terms “inflammation”, “inflammatory diseases”, “inflammatory condition” or “inflammatory process” are meant as physiological or pathological conditions, which are accompanied by an inflammatory response. The field of inflammatory diseases includes acute and chronic forms of inflammation, including inflammation as an adaptive response to insult or as a pathological process. Inflammatory disorders may include antibody-mediated events, processes driven by cells of the immune system, cytokine-induced changes, reactions to the complement system, and the scarring or functional deficit that results from these processes. The definition of inflammatory disorders also extends to sequelae of inflammation, i.e. pathology arising during or after an inflammatory process and linked to the inflammatory process. Such disorders, diseases or conditions include, but are not limited to sepsis, ischemia-reperfusion injury, cardiomyopathic disease, infectious meningitis, encephalitis, acute respiratory distress syndrome, organ/tissue transplant rejection (such as skin, kidney, heart, lung, liver, bone marrow, cornea, pancreas, small bowel), lupus, atherosclerosis, septic shock, post-ischemic leukocyte-mediated tissue damage, frost-bite injury or shock, acute leukocyte-mediated lung injury, acute pancreatitis, nephritis, asthma, traumatic shock, stroke, Crohn's disease, traumatic brain injury, acute and chronic inflammation, atopic dermatitis, uveitis, colitis, inflammatory bowel disease, rheumatoid arthritis, retinitis, psoriasis and multiple sclerosis.

The term “treatment” or “treating” is intended to include the administration of the compound of the invention to a subject for purposes which may include prophylaxis, amelioration, prevention or cure of disorders. Such treatment need not necessarily completely ameliorate the inflammatory response or other responses related to the specific disorder. Further, such treatment may be used as sole treatment or in conjunction with other traditional treatments for reducing the deleterious effects of the disease, disorder or condition as known to those of skill in the art.

The methods of the invention may be provided as a “preventive” treatment before detection of, for example, an inflammatory state, so as to prevent the disorder from developing in patients at high risk for the same, such as, for example, patients undergoing transplant.

As used through this specification and the appended claims, the singular forms “a”, “an” and “the” include the plural unless the context clearly dictates otherwise. Thus, for example, reference to “a compound” includes mixtures of such compounds, reference to “a P-selectin”, or “an L-selectin” includes reference to respective mixtures of such molecules, reference to “the formulation” or “the method” includes one or more formulations, methods and/or steps of the type described herein and/or which will become apparent to those persons skilled in the art upon reading this disclosure.

The compounds of general formula I are capable of inhibiting the interactions of HS-GAGs with VEGF, and therefore may be useful in medical applications. The inhibitory activity of Compounds 3, 4, 5 and 6 in an assay of VEGF binding to HS-GAG/heparin is described in Example 6 and Table 1.

It is further shown according to the present invention that the compounds of general formula I have anti-inflammatory activity in in vivo models of inflammation and autoimmune diseases. Thus, these compounds may be useful for prevention or treatment of inflammatory and autoimmune diseases. The anti-inflammatory activity of Compounds 3, 4, 5 and 6 in animal models of inflammation and autoimmune diseases is described in Examples 8 and 9 herein.

Example 6 and Table 1 herein describe the results of in vitro assays showing inhibition of binding of VEGF to HS-GAGs mediated by compounds of general formula I or II. Thus, the compounds of general formula I or II of the pharmaceutical compositions of the present invention inhibit the interactions of HS-GAGS with VEGF and may be useful in the treatment or prevention of diseases, disorders or conditions mediated by VEGF-HS-GAGs interaction including inflammatory and autoimmune processes or diseases exemplified by, but not restricted to, sepsis, ischemia-reperfusion injury, cardiomyopathic disease, infectious meningitis, encephalitis, acute respiratory distress syndrome, organ/tissue transplant rejection (such as skin, kidney, heart, lung, liver, bone marrow, cornea, pancreas, small bowel), lupus, atherosclerosis, septic shock, post-ischemic leukocyte-mediated tissue damage, frost-bite injury or shock, acute leukocyte-mediated lung injury, acute pancreatitis, nephritis, asthma, traumatic shock, stroke, Crohn's disease, traumatic brain injury, acute and chronic inflammation, atopic dermatitis, uveitis, colitis, inflammatory bowel disease, rheumatoid arthritis, retinitis, psoriasis and multiple sclerosis.

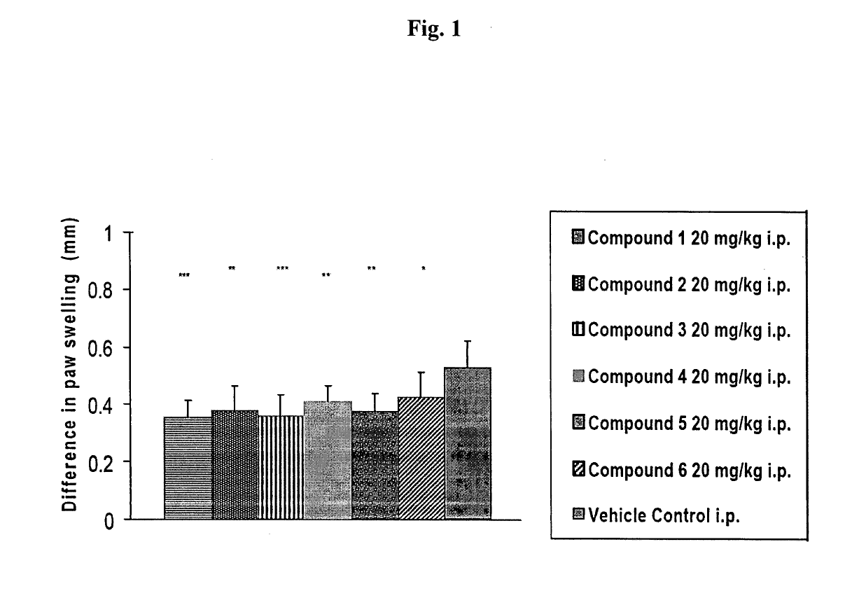

As described in Example 8, compounds of the general formula I or II inhibited carrageenan-induced paw edema (FIG. 1). Carrageenan is a family of linear sulphated polysaccharides extracted from red seaweeds and the substance is commonly used to induce inflammation in experimental animals. As shown in FIG. 1, Compounds 3-6 significantly inhibited carrageenan-induced paw edema in mice. The ability of these compounds of the invention to inhibit inflammation in vivo indicates, therefore, the potential therapeutic applications for treatment of inflammatory disorders.

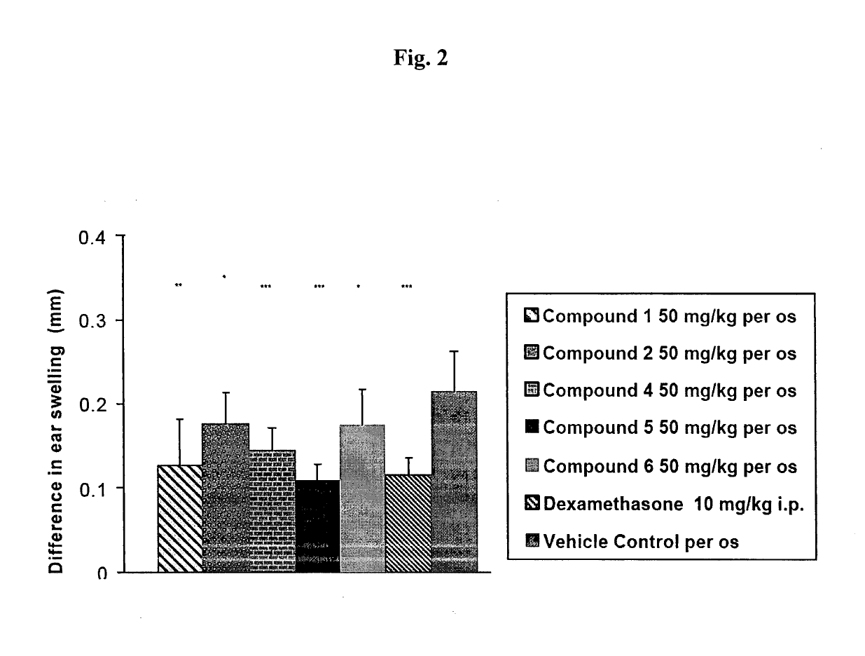

Delayed-Type Hypersensitivity (DTH) is associated mostly with T cell infiltration (Lange-Asschenfeldt et al., 2002)ir, a hallmark of autoimmune diseases. As described in Example 9, compounds of the general formula I or II inhibited DTH in experimental mice. As shown in FIG. 2, Compounds 4, 5 and 6 significantly inhibited ear swelling in the tested mice. Particularly strong response, comparable with the steroid dexamethasone, was obtained with Compound 5.

Compounds of the present invention having the desired biological activity may be modified as necessary to provide desired properties such as improved pharmacological properties.

The pharmaceutical compositions of the present invention can be formulated for administration by a variety of routes including oral, rectal, transdermal, subcutaneous, intravenous, intramuscular, and intranasal. Such compositions are prepared in a manner well known in the pharmaceutical art and comprise as an active ingredient at least one compound of formula I and/or II as described herein above, further comprising an excipient or a carrier.

For the preparation of the pharmaceutical compositions of the present invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methylcellulose. The formulations can additionally include lubricating agents such as talc, magnesium stearate, and mineral oil; wetting agents; emulsifying and suspending agents; preserving agents such as methyl- and propyl-hydroxybenzoates; sweetening agents; and flavoring agents.

The compositions of the invention can be formulated so as to provide quick, sustained or delayed release of the active ingredient after administration to the patient by employing procedures known in the art.

The compositions are preferably formulated in a unit dosage form, each dosage containing from about 0.1 to about 500 mg of a compound of formula I or II. The term “unit dosage forms” refers to physically discrete units suitable as unitary dosages for human subjects and other mammals, each unit containing a predetermined quantity of the active compound calculated to produce the desired therapeutic effect, in association with a suitable pharmaceutical excipient.

The active ingredient is effective over a wide dosage range and is generally administered in a therapeutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings; such materials include a number of polymeric acids and mixtures of polymeric acids with materials such as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. Preferably, the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

Another preferred formulation according to the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions, which can transiently open the blood-brain barrier.

Reperfusion injury is a major problem in clinical cardiology. Therapeutic agents that reduce leukocyte adherence in ischemic myocardium can significantly enhance the therapeutic efficacy of thrombolytic agents. Thrombolytic therapy with agents such as tissue plasminogen activator or streptokinase can relieve coronary artery obstruction in many patients with severe myocardial ischemia prior to irreversible myocardial cell death. However, many such patients still suffer myocardial necrosis despite restoration of blood flow. This “reperfusion injury” is known to be associated with adherence of leukocytes to vascular endothelium in the ischemic zone.

Inflammatory bowel disease (IBD) is a collective term for two similar diseases referred to as Crohn's disease and ulcerative colitis. Crohn's disease is an idiopathic, chronic ulceroconstrictive inflammatory disease characterized by sharply delimited and typically transmural involvement of all layers of the bowel wall by a granulomatous inflammatory reaction. Any segment of the gastrointestinal tract, from the mouth to the anus, may be involved, although the disease most commonly affects the terminal ileum and/or colon. Ulcerative colitis is an inflammatory response limited largely to the colonic mucosa and submucosa. Lymphocytes and macrophages are numerous in lesions of inflammatory bowel disease and may contribute to inflammatory injury. Recent evidence increasingly suggests that IBD is the result of dysfunctional immunoregulation manifested by inappropriate production of mucosal cytokines such as VEGF and b-FGF. (Kanazawa et al., 2001; 96(3): 822-828; Di Sabatino et al., 2004).

Asthma is a disease characterized by increased responsiveness of the tracheobronchial tree to various stimuli potentiating paroxysmal constriction of the bronchial airways. The stimuli cause release of various mediators of inflammation that recruit basophils, eosinophils and neutrophils, which cause inflammatory injury.

Rheumatoid arthritis (RA) is a chronic, relapsing inflammatory disease that primarily causes impairment and destruction of joints. Rheumatoid arthritis usually first affects the small joints of the hands and feet but then may involve the wrists, elbows, ankles and knees. The arthritis results from interaction of synovial cells with leukocytes that infiltrate from the circulation into the synovial lining of the joints. Angiogenesis or vascular proliferation has a pivotal role in the pathogenesis of RA by the regulation of pro and anti angiogenic factors. VEGF is one of the major angiogenic mediators found in RA synovium and implicated in the pathogenesis of joint destruction. Neutrophils (the major leucocytes in the synovial fluid of RA) are known to produce a variety of inflammatory polypeptide mediators and they also secrete VEGF. It has been also detected that VEGF has an important role in angiogenesis and endothelial migration during development of synovitis in RA (Kasama et al., 2000).

Atherosclerosis is a disease of arteries. The basic lesion, the atheroma, consists of a raised focal plaque within the intima, having a core of lipid and a covering fibrous cap. Atheromas compromise arterial blood flow and weaken affected arteries. Myocardial and cerebral infarcts are a major consequence of this disease. Macrophages and leukocytes are recruited to atheromas and contribute to inflammatory injury.

A further use of the pharmaceutical compositions according to the present invention is in treating multiple sclerosis. Multiple sclerosis is a progressive neurological autoimmune disease that is thought to be the result of a specific autoimmune reaction in which certain leukocytes initiate the destruction of myelin, the insulating sheath covering nerve fibers. Vascular permeability changes precede the development of demyelinating lesions in multiple sclerosis (MS), and vessel wall thickening and capillary proliferation are frequently seen in autopsied MS lesions. Vascular growth factors are critical for inducing such vascular changes, an increase in serum VEGF concentration might be involved in MS relapse and in the formation of longitudinally extensive spinal cord lesions (Su et al., 2006).

The pharmaceutical compositions of the present invention can be further used in the treatment of organ or graft rejection. Over recent years there has been a considerable improvement in the efficiency of surgical techniques for transplanting tissues and organs such as skin, kidney, liver, heart, lung, pancreas and bone marrow. Perhaps the principal outstanding problem is the lack of satisfactory agents for inducing immunotolerance in the recipient to the transplanted allograft or organ. When allogeneic cells or organs are transplanted into a host, the host immune system is likely to mount an immune response to foreign antigens in the transplant (host-versus-graft disease) leading to destruction of the transplanted tissue. CD8⁺ cells, CD4 cells and monocytes are all involved in the rejection of transplant tissues.

A related use of the pharmaceutical compositions according to the present invention is in modulating the immune response involved in “graft versus host” disease (GVHD). GVHD is a potentially fatal disease that occurs when immunologically competent cells are transferred to an allogeneic recipient. In this situation, the donor's immunocompetent cells may attack tissues in the recipient.

Also embodied in the present invention are methods useful for the treatment and prevention of diseases and disorders associated with angiogenesis. The term “angiogenesis” as used herein includes conditions involving abnormal neovascularization, such as tumor angiogenesis, and in opthalmologic disorders such as neovascular glaucoma, diabetic retinopathy and macular degeneration, particularly age-related macular degeneration, reperfusion of gastric ulcer, and also for contraception or for inducing abortion at early stages of pregnancy. Angiogenesis is a process of development and growth of new capillary blood vessels from pre-existing vessels, and it contributes to the development of numerous types of tumors, and the formation of metastases. Tumors cannot grow as a mass above few mm unless a new blood supply is induced. Sustained angiogenesis is one of the hallmarks of carcinogenesis. VEGF, a specific protein secreted by the tumor cells which is capable of stimulating the proliferation of capillary endothelial cells to form new vessels (angiogenesis), thus support the continued growth of tumor (Sasano et al., 2005).

HS-PGs are involved in the modulation of the neovascularization that takes place in different physiological and pathological conditions. This modulation occurs through the interaction with angiogenic growth factors including VEGF or with negative regulators of angiogenesis induced (Presta et al., 2003). Since VEGF is a crucial molecule mediating proangiogenic signals, a number of therapeutic approaches have been designed against it, such as monoclonal antibodies, small-molecule receptor kinase inhibitors, and nucleic acid inhibitors.

A further use of the pharmaceutical compositions according to the present invention is for the treatment of cancer. The term “cancer” as used herein refers to various cancer-associated conditions including both primary tumors and metastasis, tumor growth, and angiogenesis. For certain cancers to spread throughout a patient's body, a process of cell-cell adhesion, or metastasis, must take place. Specifically, cancer cells must migrate from their site of origin, the primary tumor, and gain access to a blood vessel to facilitate colonization at distant sites. A critical aspect of this process is adhesion of cancer cells, a step prior to migrating into surrounding tissue. This process can be interrupted by the administration of compounds of the invention, which generally aid in blocking cell-cell adhesion. The cancer to be treated may be a solid tumor cancer such as, but not limited to, breast, brain, lung, ovary, pancreas, kidney, liver, prostate, and esophagus cancer, or a non-solid cancer such as a leukemia, e.g. acute myeloid leukemia (AML) and adult T-cell leukemia (ATL), which involve extravasation of leukemic cells leading to organ infiltration.

It is to be understood that while the compounds according to general formula I or II of the present invention were selected for their capacity to inhibit VEGF binding to HS-GAGs, and that this property contributes to their medical activity, it cannot, however, be excluded that the compounds are also exerting their favorable medical effects, either in parallel or in tandem, through additional mechanisms of action. Thus, the skilled practitioner of this art will appreciate that one aspect of the present invention is the description of novel pharmaceutical compositions, and that Applicants intend not to be bound by a particular mechanism of action that may account for their prophylactic or therapeutic effects.

According to a further aspect, the invention relates to a method for the treatment or prevention of a disease, disorder or condition related to inhibition of VEGF binding to HS-GAGs, comprising the step of administering to a subject in need thereof a therapeutically effective amount of a pharmaceutical composition comprising at least one compound of the general formula I or II as defined herein above.

The principles of the invention, providing compounds described here for the first time with regard to their pharmaceutical uses and some novel compounds, all these compounds being capable of inhibiting HS-GAG-VEGF interactions, their pharmaceutical compositions and uses thereof according to the present invention, may be better understood with reference to the following non-limiting examples.

### EXAMPLES

The structural formulas of the Compounds 1-40 are presented in the Appendix just before the claims.

### Example 1

### General Procedure for Synthesis of Compounds of General Formula I

The synthesis of the compounds of general formula I is schematically presented in Scheme I. (Mkrtchyan et al., 2002). A compound of the formula A, wherein R₁ is as defined as in general formula I, is reacted with an acid chloride of the formula B, wherein R₂ is defined as in general formula I, in dry dioxane under reflux in the presence of triethylamine. The reaction mixture is cooled, the precipitated crystals are filtered off, washed with dioxane, hot water and methanol. The target product is re-crystallized, the crystals suspended in hot methanol and filtered off hot. The target compounds of general formula I are obtained in 60-70% yield.

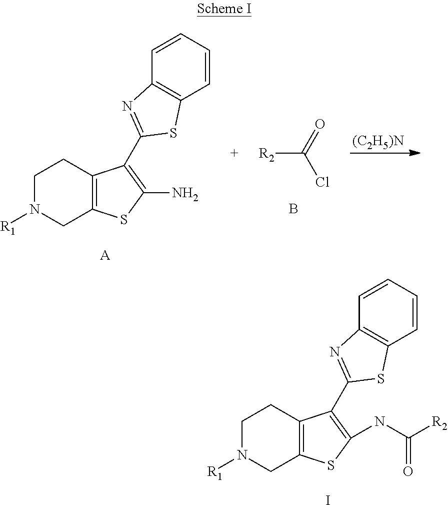

### Example 2

### Synthesis of Compound 3

To a boiling solution of 2.4 g (12 mmol) 4-(methylthio)phenylacetyl chloride in dry dioxane, 3.01 g (10 mmol) 2-amino-3-(2-benzothiazolyl)-6-methyl-4,5,6,7-tetrahydrothieno[2,3-c]pyridine and 2.2 ml of triethylamine were added. The reaction mixture was cooled, the precipitate filtered off, washed with dioxane, hot water and methanol. The precipitate was crystallized from pyridine, suspended in boiling methanol and filtered off hot. The target compound was obtained in 65% yield.

¹H NMR (DMSO-d₆, CCl₄) δ (ppm): 12.52 (s, NH), 8.00-6.90 (m, 8H), 3.89 (s, 2H), 3.52 (s, 2H), 2.93 (t, 2H), 2.78 (t, 2H), 2.47 (s, 3H), 2.43 (s, 3H).

TOF MS m/z [M+H]⁺466.

### Example 3

### Synthesis of Compound 4

To a boiling solution of 2.4 g (12 mmol) 3-(methoxy)phenoxyacetyl chloride in dry dioxane, 3.01 g (10 mmol) 2-amino-3-(2-benzothiazolyl)-6-methyl-4,5,6,7-tetrahydro-thieno[2,3-c]pyridine and 2.2 ml of triethylamine were added. The reaction mixture was cooled, the precipitate filtered off, washed with dioxane, hot water and methanol. The precipitate was crystallized from pyridine, suspended in boiling methanol and filtered off hot. The target compound was obtained in 68% yield.

¹H NMR (DMSO-d₆, CCl₄) δ (ppm): 13.33 (s, NH), 8.00-6.56 (m, 8H), 4.84 (s, 2H), 3.74 (s, 3H), 3.52 (s, 2H), 3.13 (t, 2H), 2.93 (t, 2H), 2.42 (s, 3H).

TOF MS m/z [M+H]⁺466.

### Example 4

### Synthesis of Compound 5

To a boiling solution of 2.2 g (12 mmol) benzo[b]furan-2-carbonyl chloride in dry dioxane, 3.01 g (10 mmol) 2-amino-3-(2-benzothiazolyl)-6-methyl-4,5,6,7-tetrahydrothieno[2,3-c]pyridine and 2.2 ml of triethylamine are added. The reaction mixture was cooled, the precipitate filtered off, washed with dioxane, hot water and methanol. The precipitate was crystallized from pyridine, suspended in boiling methanol and filtered off hot. The target compound was obtained in 61% yield.

¹H NMR (DMSO-d₆, CCl₄) δ (ppm): 14.10 (s, NH), 8.10-7.76 (m, 4H, benzene), 7.67 (s, 1H, furan), 7.60-7.37 (m, 4H, benzene), 3.53 (s, 2H), 2.95 (t, 2H), 2.78 (t, 2H), 2.44 (s, 3H).

TOF MS m/z [M+H]⁺446.

### Example 5

### Synthesis of Compound 6

To a boiling solution of 2.7 g (1,3-dihydro-1,3-dioxo-2H-isoindol-2)-propionyl chloride in dry dioxane, 3.01 g (10 mmol) 2-amino-3-(2-benzothiazolyl)-6-methyl-4,5,6,7-tetra-hydrothieno[2,3-c]pyridine and 2.2 ml of triethylamine were added. The reaction mixture was cooled, the precipitate filtered off, washed with dioxane, hot water and methanol. The precipitate was crystallized from pyridine, suspended in boiling methanol and filtered off hot. The target compound was obtained in 60% yield.

¹H NMR (DMSO-d₆, CCl₄) δ (ppm): 12.82 (s, NH), 8.04 0-7.37 (m, 8H, benzene), 4.03 (t, 2H), 3.55 (s, 2H), 2.95 (m, 4H), 2.76 (t, 2H), 2.40 (s, 3H).

TOF MS m/z [M+H]⁺503.

### Example 6

### In Vitro VEGF to HS-GAG Binding Assay

An in vitro assay was used to assess the ability of test compounds of general formulas I and II to inhibit the interaction of VEGF with HS-GAGs. The assay was designed to for determine the concentration required for 50% inhibition (IC-50) for each specific compound. The test compounds were purchased from Life Chemicals Ltd. (Kiev, Ukraine), and the GAG used was heparin (heparin grade I-A; Sigma) conjugated to bovine serum albumin (Sigma) and diluted 1:200 in phosphate-buffered saline (PBS; pH 7.3). The heparin was added to a 96-well polystyrene ELISA plate (NUNC; 0.1 ml per well) and incubated overnight at 4° C. Following the incubation, the plate was washed thoroughly, by immersion, with de-ionized water and then with PBS (pH 7.3) containing Tween 20 (Sigma, 0.05%). The ELISA plate was then blocked with BSA (ICN, 3%, 200 μl per well) for 1 hour at room temperature. Following blocking, the plate was washed with de-ionized water, and then twice with PBS (pH 7.3) containing Tween 20. Recombinant human VEGF (Research and Development Systems) dissolved in PBS supplemented with BSA (0.1%) was added to the ELISA plate. The test compounds were dissolved in DMSO (MERCK), diluted in PBS and added to the wells at concentrations ranging from 0.01 to 200 μM to obtain a final volume 100 μl per well, and incubated for 60 minutes at room temperature with shaking. Following incubation, the plate was washed with de-ionized water and PBS (pH 7.3) containing Tween 20. Anti-human VEGF (1:2000; Research and Development Systems) diluted in PBS supplemented with BSA (1%) was added to the ELISA plate (100 per well) and incubated for 60 minutes at room temperature with shaking. Following incubation, the plate was washed with de-ionized water and then with PBS (pH 7.3) containing Tween 20. Streptavidin-HRP (1:200; Research and Development Systems Cat. No. DY998) diluted in PBS supplemented with BSA (1%) was added to the ELISA plate (100 per well) and incubated for 20 minutes at room temperature with shaking. The plate was then washed with de-ionized water and twice with PBS (pH 7.3) containing Tween 20. The peroxidase substrate chromogen tetramethyl benzidine (TMB; Dako) was added (100 μl per well) to the ELISA plate and incubated at room temperature. After 15 minutes, ELISA Stop Solution (hydrochloric acid 1N, sulfuric acid 3N) was added (100 μl per well) to stop the peroxidase catalyzed colorimetric reaction. The Optical Density (OD) of the samples was measured at 450 nm using an ELISA plate reader (Dynatech MR5000). Data were analyzed with Graphpad Prism software and IC-50 values were determined.

As shown in Table I, the compounds 1-40 of the general formulas I and II had inhibitory activity in the above assay. Percent inhibition at 30 μM concentration is shown and for selected compounds IC-50 was established.

TABLE I
Results of in vitro inhibition assay fro VEGF binding to heparin

| Compound | % Inhibition | IC- 50 |
|---|---|---|
| No. | at 30 μM | [μM] |
| 1 | 82 | 0.22 |
| 2 | 53 | 1.7 |
| 3 | 60 | N.D.* |
| 4 | 65 | 1.8 |
| 5 | 81 | 0.14 |
| 6 | 29 | N.D. |
| 7 | 70 | 1.7 |
| 8 | 82 | 0.4 |
| 9 | 70 | 0.50 |
| 10 | 71 | N.D. |
| 11 | 41 | N.D. |
| 12 | 62 | N.D. |
| 13 | 64 | N.D. |
| 14 | 73 | 0.20 |
| 15 | 43 | N.D. |
| 16 | 61 | N.D. |
| 17 | 77 | 0.30 |
| 18 | 44 | N.D. |
| 19 | 49 | N.D. |
| 20 | 77 | 0.24 |
| 21 | 79 | 0.90 |
| 22 | 39 | N.D. |
| 23 | 57 | N.D. |
| 24 | 52 | N.D. |
| 25 | 55 | N.D. |
| 26 | 36 | N.D. |
| 27 | 55 | N.D. |
| 28 | 35 | N.D. |
| 29 | 72 | 1.30 |
| 30 | 29 | N.D. |
| 31 | 37 | N.D. |
| 32 | 31 | N.D. |
| 33 | 50 | N.D. |
| 34 | 77 | 0.20 |
| 35 | 37 | N.D. |
| 36 | 31 | N.D. |
| 37 | 75 | 0.35 |
| 38 | 50 | N.D. |
| 39 | 42 | N.D. |
| 40 | 41 | N.D. |

*N.D. = Not Determined.

### Example 7

### Formulations

The pharmaceutical compositions of the present invention are illustrated by the following formulation examples:

### (i) Formulation 1

Hard gelatin capsules containing the following ingredients are prepared:

| Ingredient | Quantity (mg/capsule) |
|---|---|
| Active Ingredient | 30.0 |
| Starch | 305.0 |
| Magnesium stearate | 5.0 |

The above ingredients are mixed and filled into hard gelatin capsules in 340 mg quantities.

### (ii) Formulation 2

A tablet is prepared using the ingredients below:

| Ingredient | Quantity (mg/tablet) |
|---|---|
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

(iii) Formulation 3

A dry powder inhaler formulation is prepared containing the following components:

| Ingredient | Weight % |
|---|---|
| Active Ingredient | 5.0 |
| Lactose | 95.0 |

The active ingredient is mixed with the lactose and the mixture is added to a dry powder inhaling-appliance.

### (iv) Formulation 4

Tablets, each containing 30 mg of active ingredient, are prepared as follows:

| Ingredient | Quantity (mg/tablet) |
|---|---|
| Active Ingredient | 30.0 |
| Starch | 45.0 |
| Microcrystalline cellulose | 35.0 |
| Polyvinylpyrrolidone | 4.0 |
| (as 10% solution in water) | |
| Sodium carboxymethyl starch | 4.5 |
| Magnesium stearate | 0.5 |
| Talc | 1.0 |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### (v) Formulation 5

Capsules, each containing 40 mg of the active ingredient, are made as follows:

| Ingredient | Quantity (mg/capsule) |
|---|---|
| Active Ingredient | 40.0 |
| Starch | 109.0 |
| Magnesium stearate | 1.0 |

The active ingredient, starch, and magnesium stearate are blended, passed through a No. 20 mesh U.S. sieve, and filled into hard gelatin capsules in 150 mg quantities.

### (vi) Formulation 6

Suppositories, each containing 25 mg of active ingredient, are made as follows:

| Ingredient | Quantity (mg) |
|---|---|
| Active Ingredient | 25.0 |
| Saturated fatty acid glycerides | 2000.0 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

(vii) Formulation 7

Suspensions, each containing 50 mg of an active ingredient per 5.0 ml dose, are made as follows:

| Ingredient | Quantity (mg) | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | mg |
| Purified water | to 5.0 | ml |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

(viii) Formulation 8

Capsules, each containing 15 mg of an active ingredient, are made as follows:

| Ingredient | Quantity (mg/capsule) |
|---|---|
| Active Ingredient | 15.0 |
| Starch | 407.0 |
| Magnesium stearate | 3.0 |

The active ingredient, cellulose, starch, and magnesium stearate are blended, passed through a No. 20 mesh U.S. sieve, and filled into hard gelatin capsules in 425 mg quantities.

### (ix) Formulation 9

An intravenous formulation is prepared as follows:

| Ingredient | Quantity |
|---|---|
| Active Ingredient | 250.0 mg |
| Isotonic saline | 1000 ml |

The active ingredient is thoroughly dissolved in the isotonic solution to obtain the aqueous formulation suitable for injection.

### (x) Formulation 10

A topical formulation is prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solidified.

### Example 8

### Carrageenan-Induced Paw Edema

Acute edema was induced in the left hind paw of BALB/c mice (10-12 mice/group) by injecting 0.02 ml of freshly prepared solution of 2% carrageenan (Sigma) under the plantar region of right hind paw (Tones et al., 2000). Carrageenan was injected 60 min after administration of the test compounds. The right paw received 0.02 ml of saline, which served as a control. The paw thickness was measured at 2, 4 and 24 hours after carrageenan challenge using a Mitutoyo engineer's micrometer. The difference between right and left pad was expressed as mean±SEM. Test compounds were injected intraperitoneally (i.p.) (20 mg/kg) in 0.5% Tween 80 in saline and vehicle control group received 0.5% Tween 80 in saline.

As shown in FIG. 1, intraperitoneal (i.p.) administration of test Compounds 1-6 at resulted in reduced paw swelling 4 hours after induction with carrageenan. Compound 1 reduced paw edema by 33% and the reduction was statistically significant as determined by Student's t-test with p>0.001. Compound 2 reduced paw edema by 28.1% (Student's t-test p>0.01); Compound 3 reduced paw edema by 32.1% (Student's t-test p>0.001); Compound 4 reduced paw edema by 22.8% (Student's t-test p>0.01); Compound 5 reduced paw edema by 29.2% (Student's t-test p>0.01); and Compound 6 reduced paw edema by 19.5% (Student's t-test p>0.05). In a separate experiment (data not shown), Compound 7 reduced paw edema at 50 mg/kg dose by 18.5% (Student's t-test p>0.05).

### Example 9

### Delayed-Type Hypersensitivity (DTH)

BALB/c mice (Velaz, Prague, Czech Republic; 8 week-old; 10-15 animals per group) were sensitized by topical application of a 2% oxazolone (4-ethoxymethylene-2-phenyl-2-oxazolin-5-one; Sigma, St Louis, Mo.) solution in acetone/olive oil (4:1 vol/vol) to a shaved abdomen area (50 μl) and to each paw (5 μl)(Lange-Asschenfeldt et al., 2002). Topical oxazolone administration induces immunomodulatory activity. Five days after sensitization, right ears were challenged by topical application of 10 μl of a 1% oxazolone solution, whereas left ears were treated with vehicle alone. Test compounds were administered 1 hour prior to challenge. The extent of inflammation was measured 24 hours and 48 hours after challenge, using the mouse ear-swelling test. Animals were numbered (tail marking) and weighed and the thickness of both ears was recorded with a constant-loading dial micrometer (Mitutoyo, Tokyo). The unpaired Student t-test was used for statistical analyses. Test compounds were administered per os at 50 mg/kg as a suspension with 0.5% methylcellulose. The vehicle control group received the same volume of 0.5% methylcellulose.

As shown in FIG. 2, oral administration of test Compounds 1, 2, 4, 5, and 6 resulted in significant reduction in ear swelling after 48 hours. Compound 1 reduced ear swelling by 41.4% and the reduction was statistically significant as determined by Student's t-test with p>0.01. Compound 2 reduced ear swelling by 18.1% (Student's t-test p>0.05); Compound 4 reduced ear swelling by 33.5% (Student's t-test p>0.001); Compound 5 reduced ear swelling by 49.3% (Student's t-test p>0.001); and Compound 6 reduced ear swelling by 19.1% (Student's t-test p>0.05). For comparison, dexamethasone, a steroidal positive control test compound administered i.p. gave 46.5% reduction in ear swelling. In a separate experiment (data not shown), Compound 7, administered per os at 50 mg/kg, reduced ear swelling after 48 hours by 32.7% (Student's t-test p>0.001).

### APPENDIX

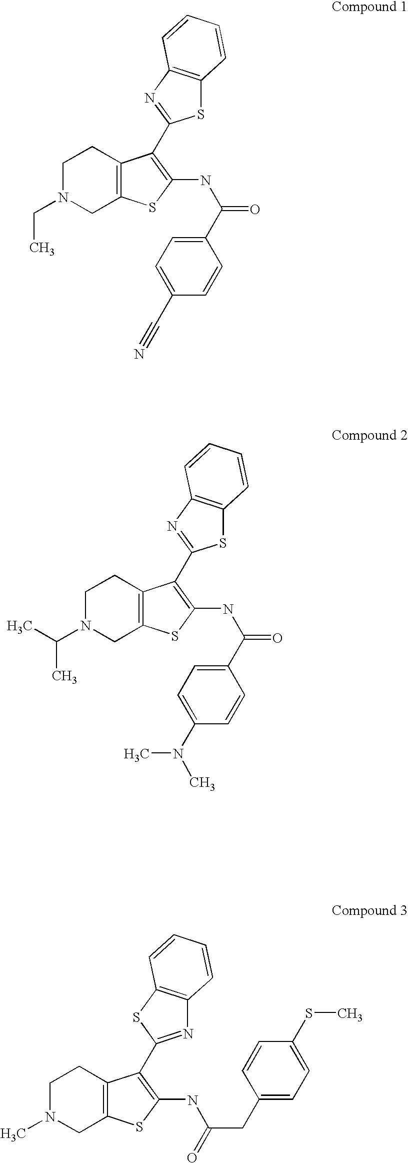

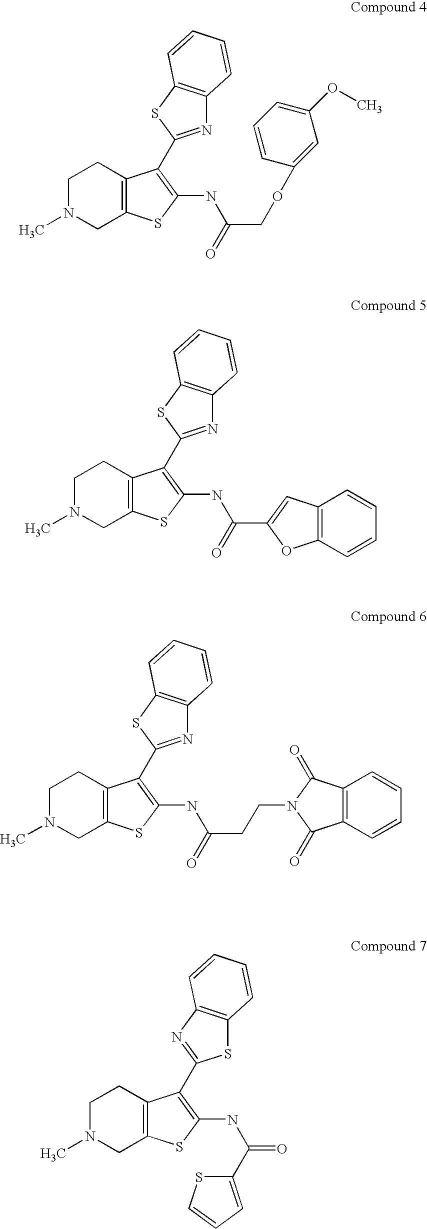

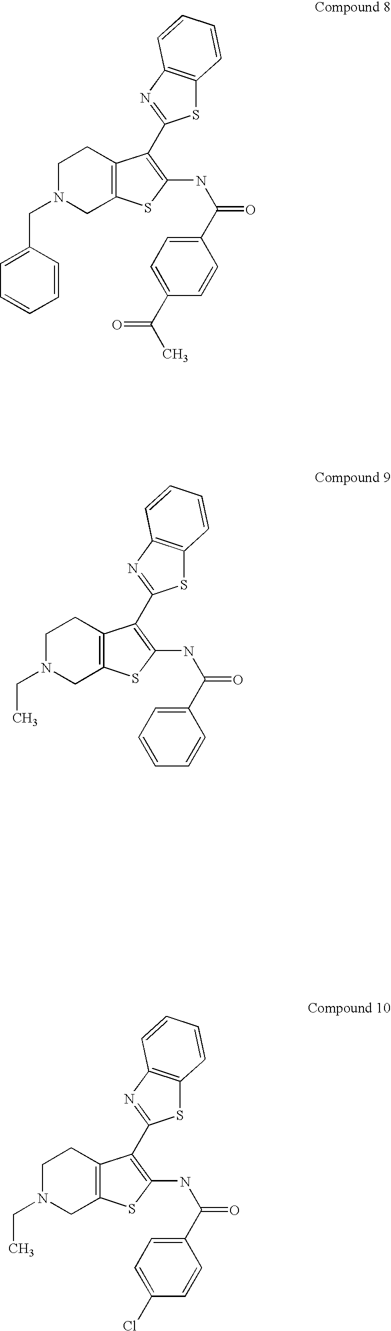

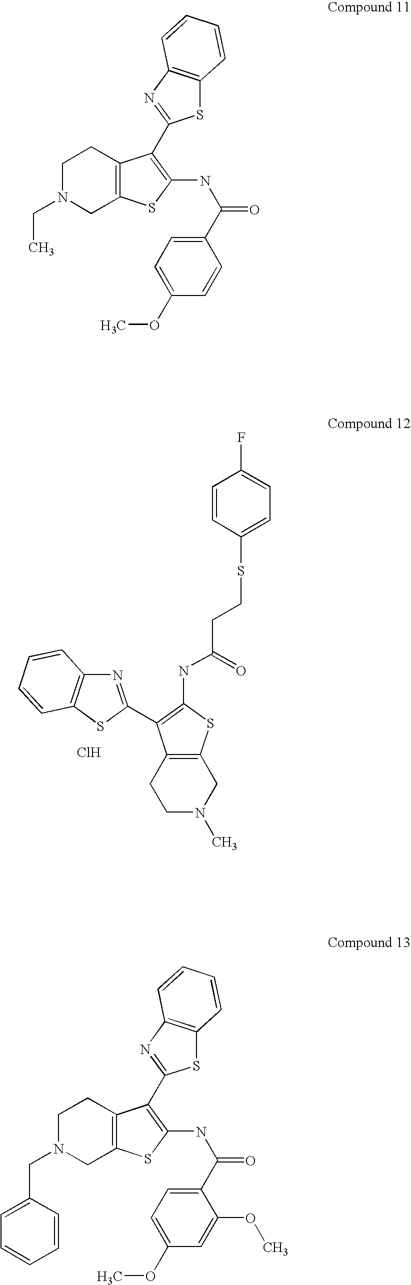

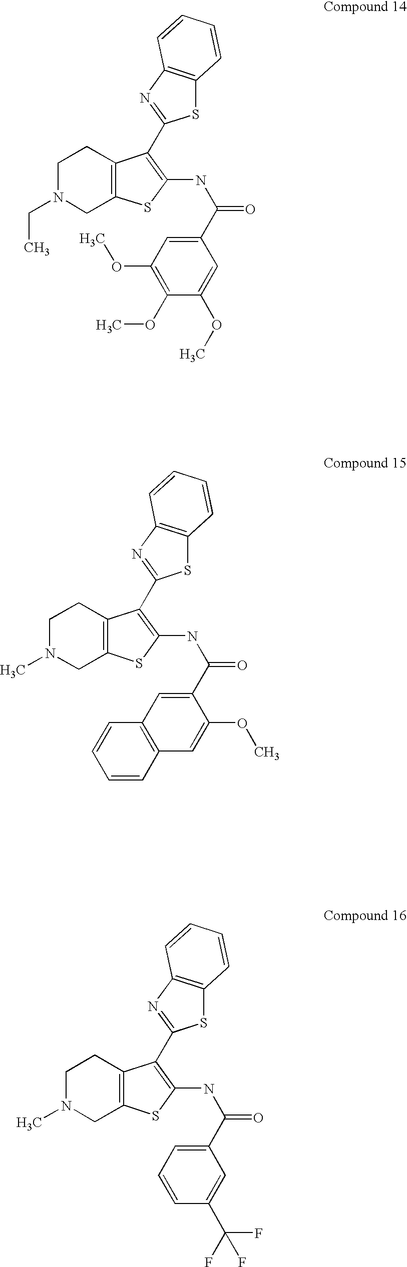

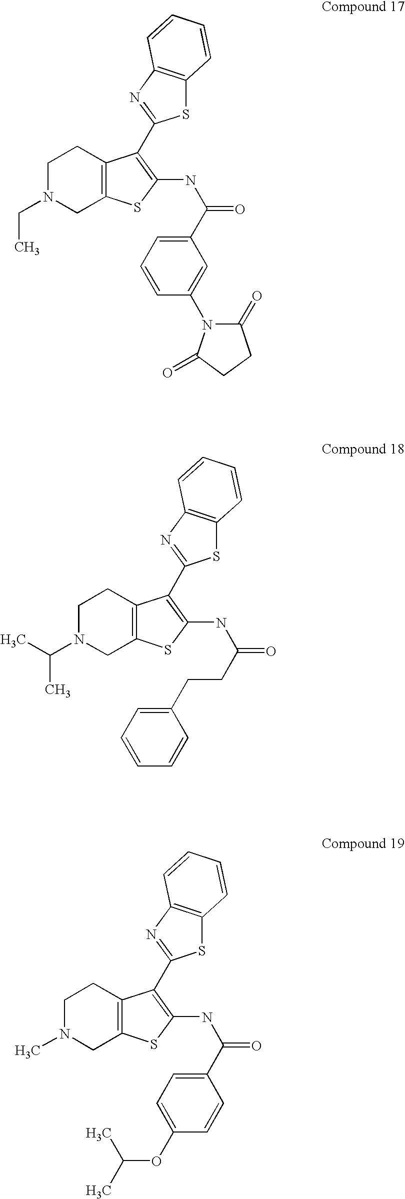

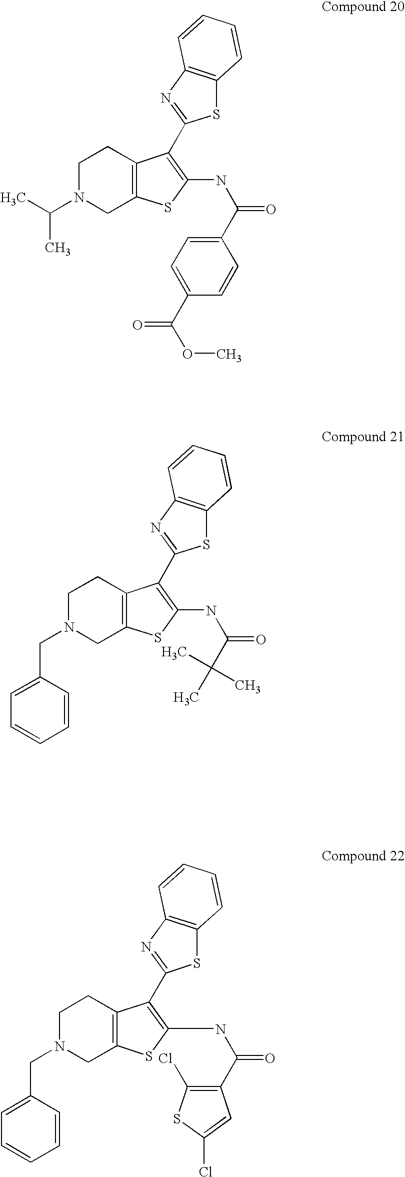

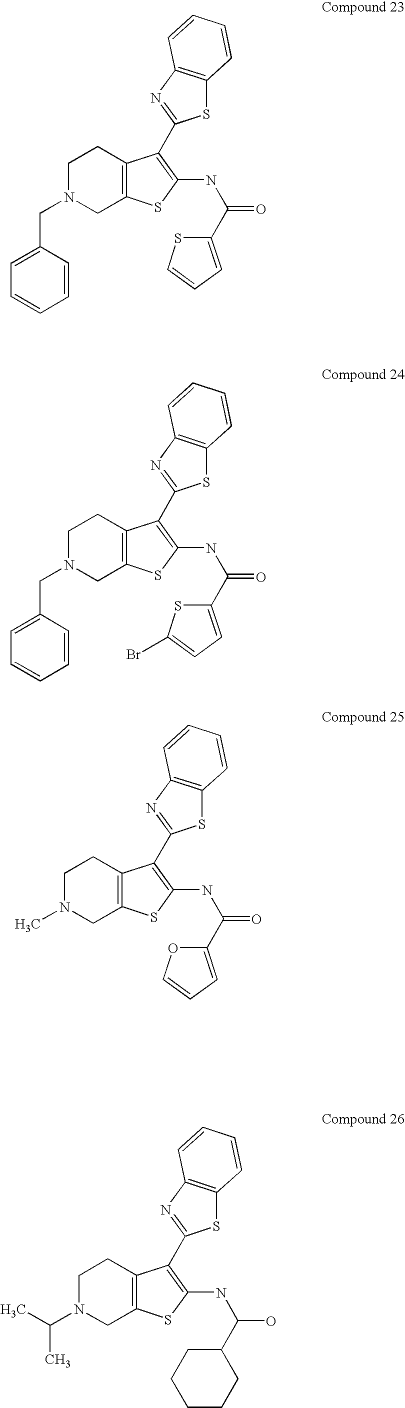

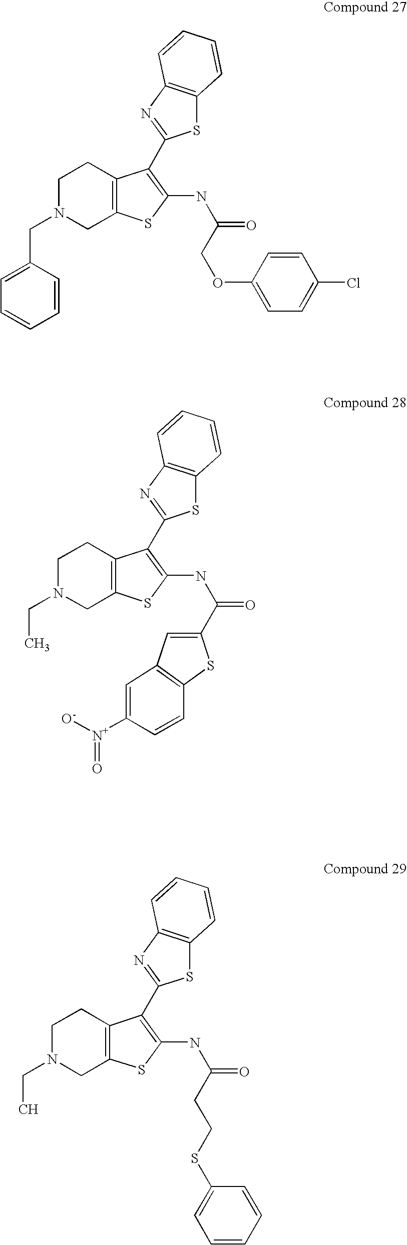

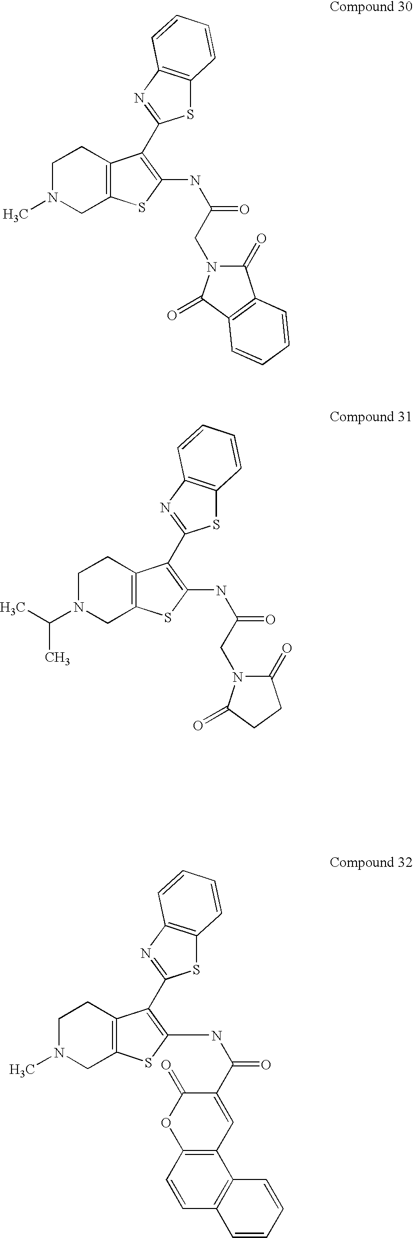

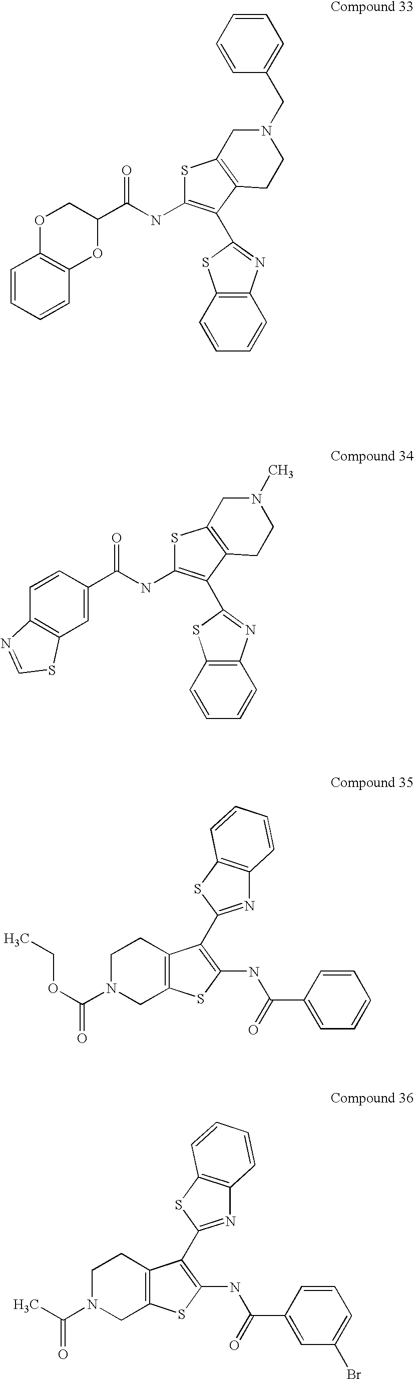

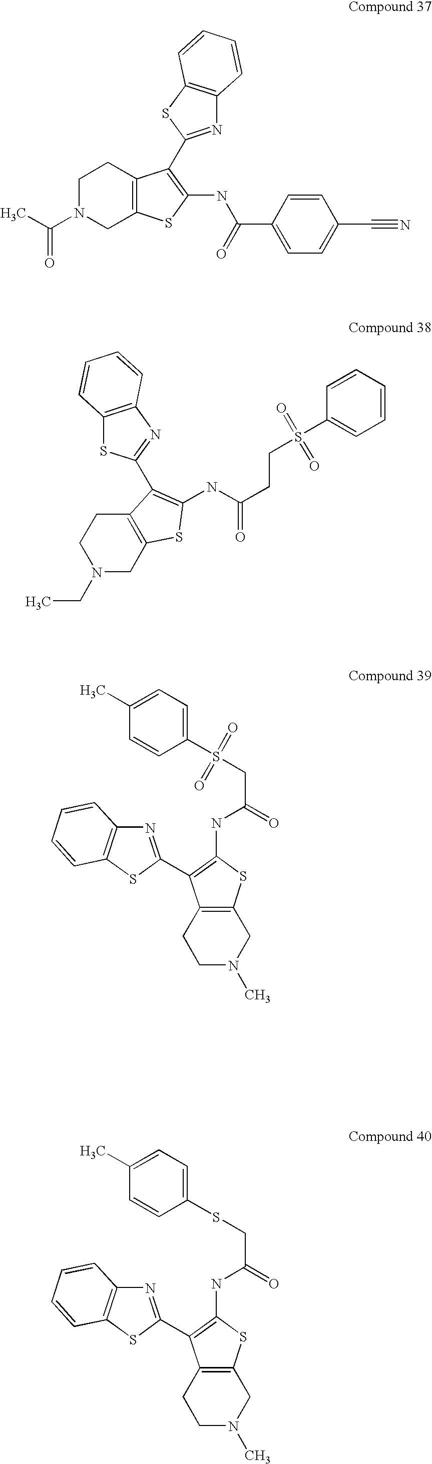

### REFERENCES

Christopher J. R. et al., (2006), J. Biol. Chem. 281(3):1731-1740.
Di Sabatino A. et al., (2004), Inflamm. Bowel Dis. 10(5):573-577.
Fujita M. et al. (2002a), Bioorg. Med. Chem. Lett 12:1607-1611.
Fujita M. et al. (2002b), Bioorg. Med. Chem. Lett 12: 1897-1900.
Johnson et al. (2004), Biochem Soc Trans., 32(Pt 2):366-77.
Kanazawa S. et al., (2001), Am. J. Gastroenterol. 96(3):822-828.
Kasama K. et al., (2000), Clin. Exp. Immunol. 121:533-538.
Kim I. et al., (2001), J. Biol. Chem. 276: 7614-7620.
Lange-Asschenfeldt et al., (2002) Blood 99:538-545.
Maruotti N. et al. (2006), Histol. Histopathol. 21(5):557-566.
Melder R. J. et al., (1996), Nat. Med. 2:992-997.
Mkrtchyan A. P., Noravyan A. S., Petrosyan V. M. (2002), Chemistry of Hetrocyclic Compounds 38(2):238-241 (translated from Khimiya Geterotsiklicheskikh Soedinenii, No. 2, 261-264, 2002).
Navarro C. et al. (2002), Lupus 11(1):21-24.
Presta M. et al. (2003), Curr. Pharm. Des. 9(7):553-566.
Rohit K. et al. (2005), Circulation 112:1813-1824.
Sasano H. et al. (2005), Biomed Pharmacother 59(2):334-336
Sasisekharan and Venkataraman (2000), Current Opinion in Chem. Biol. 4:626-631.
Su J. J. et al., (2006), J Neurol Sci. 243(1-2):21-30.
Tanaka et al., (1998), Proc. Assoc. Am. Physicians 110:118-125.
Torres et al. (2000), Eur. J. of Pharmacol. 408:199-211.
Tsuchihashi S. et al., (2006) Am. J. Pathol. 168(2): 695-705.
Wayne J. F et al, (1998) Structure 15(6):637-648.

1. A compound of the general formula I: 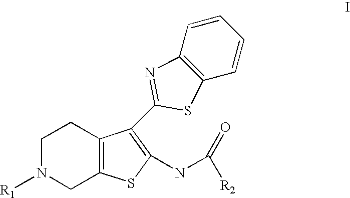
wherein
R₁ is optionally substituted hydrocarbyl, alkylcarbonyl or alkoxycarbonyl;
R₂ is [(4-methylthio)phenyl]methyl, (3-methoxyphenoxy)methyl, benzo[b]fur-2-yl and 1,3-dihydro-1,3-dioxo-2H-isoindol-2-yl-ethyl.

2. The compound according to claim 1, which is N-[3-(2-benzothiazolyl)-4,5,6,7-tetrahydro-6-methylthieno[2,3-c]pyridin-2-yl]-2-(4-methylthiophenyl)acetamide (Compound 3).

3. The compound according to claim 1, which is N-[3-(2-benzothiazolyl)-4,5,6,7-tetrahydro-6-methyl thieno[2,3-c]pyridin-2-yl]-2-(3-methoxyphenoxy)acetamide (Compound 4).

4. The compound according to claim 1, which is N-[3-(2-benzothiazolyl)-4,5,6,7-tetrahydro-6-methyl thieno[2,3-c]pyridin-2-yl]-benzo[b]furan-2-carboxamide (Compound 5).

5. The compound according to claim 1, which is N-[3-(2-benzothiazolyl)-4,5,6,7-tetrahydro-6-methylthieno[2,3-c]pyridin-2-yl]-3-(1,3-dihydro-1,3-dioxo-2H-isoindol-2-yl) propanamide (Compound 6).

6. A pharmaceutical composition comprising a pharmaceutically acceptable diluent or carrier and at least one compound of the general formula II: 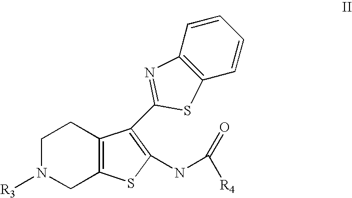
wherein:
R₃ is optionally substituted hydrocarbyl, alkylcarbonyl or alkoxycarbonyl;
R₄ is optionally substituted hydrocarbyl, aryloxyalkyl, arylthioalkyl, arylsulfonylalkyl and heterocyclyl;
and pharmaceutically acceptable salts thereof.

7. A pharmaceutical composition according to claim 6, wherein:
R₃ is (C₁-C₆)alkyl, (C₆-C₂₀)aryl, (C₆-C₂₀)aryl(C₁-C₆)alkyl, (C₁-C₆)alkyl carbonyl or (C₁-C₆)alkoxycarbonyl; and
R₄ is (C₁-C₆)alkyl, (C₃-C₈)cycloalkyl, (C₆-C₂₀)aryl, (C₆-C₂₀)aryl(C₁-C₆)alkyl, (C₆-C₂₀)aryloxy(C₁-C₆)alkyl, (C₆-C₂₀)arylthio(C₁-C₆)alkyl, (C₆-C₂₀)arylsulfonyl(C₁-C₆)alkyl, (C₄-C₁₂)heteroaryl or (C₆-C₁₀)bicyclic heterocyclyl;
wherein each (C₁-C₆)alkyl or (C₆-C₂₀)aryl either alone or as part of an aralkyl, alkylcarbonyl, alkoxycarbonyl, aryloxyalkyl, arylthioalkyl and arylsulfonyl-alkyl moiety and any heteroaryl or heterocyclyl, may be optionally substituted by one to three radicals selected from the group consisting of halogen, cyano, amino, hydroxy, oxo, nitro, (C₁-C₆)alkyl, (C₁-C₆)haloalkyl, (C₃-C₈)cycloalkyl, (C₁-C₆)alkoxy, (C₁-C₆)alkylcarbonyl, (C₁-C₆)alkoxycarbonyl, (C₁-C₆)alkylsulfonyl, (C₁-C₆)alkylthio and heterocyclyl.

8. The pharmaceutical composition according to claim 7, wherein
R₃ is a straight or branched (C₁-C₃)alkyl, benzyl, acetyl or ethoxycarbonyl; and
R₄ is selected from the group consisting of:
straight or branched (C₁-C₄)alkyl, unsubstituted or substituted by (C₃-C₆)cycloalkyl, 2,5-dioxo-pyrrolidin-1-yl or 1,3-dioxo-isoindol-2-yl;
(C₃-C₆)cycloalkyl;
phenyl, unsubstituted or substituted by one to three radicals selected from the group consisting of halogen, cyano, dimethylamino, trifluoromethyl, (C₁-C₃)alkoxy, (C₁-C₃)alkylcarbonyl, (C₁-C₃)alkoxycarbonyl and 2,5-dioxo-pyrrolidin-2-yl;
1-naphthyl or 2-naphthyl, substituted by (C₁-C₃)alkoxy; phenyl(C₁-C₃)alkyl, unsubstituted or substituted by methylthio on the phenyl moiety;
phenoxy(C₁-C₃)alkyl, unsubstituted or substituted on the phenyl moiety by one to three radicals selected from the group consisting of halogen, alkoxy or acetyl;
phenylthio(C₁-C₃)alkyl, unsubstituted or substituted on the phenyl moiety by one to three (C₁-C₃)alkyl radicals;
phenylsulfonyl(C₁-C₃)alkyl, unsubstituted or substituted on the phenyl moiety by one to three (C₁-C₃)alkyl radicals;
heteroaryl selected from the group consisting of furyl and thienyl, unsubstituted or substituted by one or two halogen atoms;
benzofuryl; benzothienyl substituted by a nitro radical; and
benzthiazolyl; and
heterocyclyl selected from the group consisting of 1,3-benzodioxolyl; 4-oxo-benzopyranyl, 3-oxo-3H-naphto[2,1-b]pyranyl and 1,4-benzodioxinyl.

9. The pharmaceutical composition according to claim 6 wherein said compound of formula II is selected from the group consisting of:
N-[3-(2-benzothiazolyl)-4,5,6,7-tetrahydro-6-ethylthieno[2,3-c]pyridin-2-yl]-4-cyanobenzamide (Compound 1);
N-[3-(2-benzothiazolyl)-4,5,6,7-tetrahydro-6-(1-methylethyl)thieno[2,3-c]pyridin-2-yl]-4-(dimethylamino)benzamide (Compound 2);
N-[3-(2-benzothiazolyl)-4,5,6,7-tetrahydro-6-methylthieno[2,3-c]pyridin-2-yl]-2-(4-methylthiophenyl)acetamide (Compound 3);
N-[3-(2-benzothiazolyl)-4,5,6,7-tetrahydro-6-methylthieno[2,3-c]pyridin-2-yl]-2-(3-methoxyphenoxy)acetamide (Compound 4);
N-[3-(2-benzothiazolyl)-4,5,6,7-tetrahydro-6-methylthieno[2,3-c]pyridin-2-yl]-benzo[b]furan-2-carboxamide (Compound 5);
N-[3-(2-benzothiazolyl)-4,5,6,7-tetrahydro-6-methylthieno[2,3-c]pyridin-2-yl]-3-(1,3-dihydro-1,3-dioxo-2H-isoindol-2-yl)propanamide (Compound 6);
N-[3-(2-benzothiazolyl)-4,5,6,7-tetrahydro-6-methylthieno[2,3-c]pyridin-2-yl]-2-thiophene-carboxamide (Compound 7);
N-[3-(2-benzothiazolyl)-4,5,6,7-tetrahydro-6-phenylmethylthieno[2,3-c]pyridin-2-yl]-4-acetylbenzamide (Compound 8);
N-[3-(2-benzothiazolyl)-4,5,6,7-tetrahydro-6-ethylthieno[2,3-c]pyridin-2-yl]-benzamide (Compound 9);
N-[3-(2-benzothiazolyl)-4,5,6,7-tetrahydro-6-ethylthieno[2,3-c]pyridin-2-yl]-(4-chloro)benzamide (Compound 10);
N-[3-(2-benzothiazolyl)-4,5,6,7-tetrahydro-6-ethylthieno[2,3-c]pyridin-2-yl]-4-methoxybenzamide (Compound 11);
N-[3-(2-benzothiazolyl)-4,5,6,7-tetrahydro-6-methylthieno[2,3-c]pyridin-2-yl]-3-[(4-fluorophenyl)thio]propanamide (Compound 12);
N-[3-(2-benzothiazolyl)-4,5,6,7-tetrahydro-6-phenylmethylthieno[2,3-c]pyridin-2-yl]-(2,4-dimethoxy)benzamide (Compound 13);
N-[3-(2-benzothiazolyl)-4,5,6,7-tetrahydro-6-ethylthieno[2,3-c]pyridin-2-yl]-(3,4,5-trimethoxy)benzamide (Compound 14);
N-[3-(2-benzothiazolyl)-4,5,6,7-tetrahydro-6-methylthieno[2,3-c]pyridin-2-yl]-3-methoxy-2-naphthalene-carboxamide (Compound 15);
N-[3-(2-benzothiazolyl)-4,5,6,7-tetrahydro-6-methylthieno[2,3-c]pyridin-2-yl]-3-trifluoromethylbenzamide (Compound 16);
N-[3-(2-benzothiazolyl)-4,5,6,7-tetrahydro-6-ethylthieno[2,3-c]pyridin-2-yl]-3-(2,5-dioxo-1-pyrrolidinyl)benzamide (Compound 17);
N-[3-(2-benzothiazolyl)-4,5,6,7-tetrahydro-6-(1-methylethyl)thieno[2,3-c]pyridin-2-yl]-3-phenylpropanamide (Compound 18);
N-[3-(2-benzothiazolyl)-4,5,6,7-tetrahydro-6-methylthieno[2,3-c]pyridin-2-yl]-4-(1-methylethoxy)benzamide (Compound 19);
N-[3-(2-benzothiazolyl)-4,5,6,7-tetrahydro-6-(1-methylethyl)thieno[2,3-c]pyridin-2-yl]amino]carbonyl]benzoic acid methyl ester (Compound 20);
N-[3-(2-benzothiazolyl)-4,5,6,7-tetrahydro-6-phenylmethyllthieno[2,3-c]pyridin-2-yl]-2,2-dimethylpropanamide (Compound 21);
N-[3-(2-benzothiazolyl)-4,5,6,7-tetrahydro-6-phenylmethyllthieno[2,3-c]pyridin-2-yl]-3-(2,5-dichloro)thiophene-carboxamide (Compound 22);
N-[3-(2-benzothiazolyl)-4,5,6,7-tetrahydro-6-phenylmethylthieno[2,3-c]pyridin-2-yl]-2-thiophene-carboxamide (Compound 23);
N-[3-(2-benzothiazolyl)-4,5,6,7-tetrahydro-6-phenylmethylthieno[2,3-c]pyridin-2-yl]-2-(5-bromo)thiophene-carboxamide (Compound 24);
N-[3-(2-benzothiazolyl)-4,5,6,7-tetrahydro-6-methylthieno[2,3-c]pyridin-2-yl]-2-furan-carboxamide (Compound 25);
N-[3-(2-benzothiazolyl)-4,5,6,7-tetrahydro-6-(1-methylethyl)thieno[2,3-c]pyridin-2-yl]-cyclohexylcarboxamide (Compound 26);
N-[3-(2-benzothiazolyl)-4,5,6,7-3-tetrahydro-6-phenylmethylthieno[2,3-c]pyridin-2-yl]-2-(4-chlorophenoxy)acetamide (Compound 27);
N-[3-(2-benzothiazolyl)-4,5,6,7-tetrahydro-6-ethylthieno[2,3-c]pyridin-2-yl]-benzo[b]thiophene-5-nitro-2-carboxamide (Compound 28);
N-[3-(2-benzothiazolyl)-4,5,6,7-tetrahydro-6-ethylthieno[2,3-c]pyridin-2-yl]-3-(phenylthio)propanamide (Compound 29);
N-[3-(2-benzothiazolyl)-4,5,6,7-tetrahydro-6-methylthieno[2,3-c]pyridin-2-yl]-1,3-dihydro-1,3-dioxo-2H-isoindol-2-yl-acetamide (Compound 30);
N-[3-(2-benzothiazolyl)-4,5,6,7-tetrahydro-6-(methylethyl)thieno[2,3-c]pyridin-2-yl]-(2,5-dioxo-1-pyrrolidinyl)acetamide (Compound 31);
N-[3-(2-benzothiazolyl)-4,5,6,7-tetrahydro-6-methylthieno[2,3-c]pyridin-2-yl]-3-oxo-3H-naphtho[2,1-b]pyran-2-carboxamide (Compound 32);
N-[3-(2-benzothiazolyl)-4,5,6,7-tetrahydro-6-phenylmethylthieno[2,3-c]pyridin-2-yl]-2,3-dihydro-1,4-benzodioxin-2-carboxamide (Compound 33);
N-[3-(2-benzothiazolyl)-4,5,6,7-tetrahydro-6-methylthieno[2,3-c]pyridin-2-yl]-6-benzothiazole-carboxamide (Compound 34);
N-[3-(2-benzothiazolyl)-4,5,6,7-tetrahydro-6-ethoxycarbonylthieno[2,3-c]pyridin-2-yl]benzamide (Compound 35);
N-[3-(2-benzothiazolyl)-4,5,6,7-tetrahydro-6-acetylthieno[2,3-c]pyridin-2-yl]-3-bromo-benzamide (Compound 36);
N-[3-(2-benzothiazolyl)-4,5,6,7-tetrahydro-6-acetylthieno[2,3-c]pyridin-2-yl]-4-cyano-benzamide (Compound 37);
N-[3-(2-benzothiazolyl)-4,5,6,7-tetrahydro-6-ethylthieno[2,3-c]pyridin-2-yl]-3-(phenylsulfonyl)propanamide (Compound 38);l
N-[3-(2-benzothiazolyl)-4,5,6,7-tetrahydro-6-methylthieno[2,3-c]pyridin-2-yl]-(4-methylphenylsulfonyl)acetamide (Compound 39); and
N-[3-(2-benzothiazolyl)-4,5,6,7-tetrahydro-6-methylthieno[2,3-c]pyridin-2-yl]-(4-methylphenyl)thioacetamide (Compound 40).

10. The pharmaceutical composition according to claim 6 comprising as active agent the compound N-[3-(2-benzothiazolyl)-4,5,6,7-tetrahydro-6-methylthieno[2,3-c]pyridin-2-yl]-2-(4-methylthiophenyl)acetamide (Compound 3).

11. The pharmaceutical composition according to claim 6 comprising as active agent the compound N-[3-(2-benzothiazolyl)-4,5,6,7-tetrahydro-6-methylthieno[2,3-c]pyridin-2-yl]-2-(3-methoxyphenoxy)acetamide (Compound 4).

12. The pharmaceutical composition according to claim 6 comprising as active agent the compound N-[3-(2-benzothiazolyl)-4,5,6,7-tetrahydro-6-methylthieno[2,3-c]pyridin-2-yl]-benzo[b]furan-2-carboxamide (Compound 5).

13. The pharmaceutical composition according to claim 6 comprising as active agent the compound N-[3-(2-benzothiazolyl)-4,5,6,7-tetrahydro-6-methylthieno[2,3-c]pyridin-2-yl]-3-(1,3-dihydro-1,3-dioxo-2H-isoindol-2-yl) propanamide (Compound 6).

14. The pharmaceutical composition according to claim 6, for the treatment or prevention of inflammatory or autoimmune diseases, disorders or conditions, selected from the group consisting of sepsis, ischemia-reperfusion injury, cardiomyopathic disease, infectious meningitis, encephalitis, acute respiratory distress syndrome, organ/tissue transplant rejection (such as skin, kidney, heart, lung, liver, bone marrow, cornea, pancreas, small bowel), lupus, atherosclerosis, septic shock, post-ischemic leukocyte-mediated tissue damage, frost-bite injury or shock, acute leukocyte-mediated lung injury, acute pancreatitis, nephritis, asthma, traumatic shock, stroke, Crohn's disease, traumatic brain injury, acute and chronic inflammation, atopic dermatitis, uveitis, colitis, inflammatory bowel disease, rheumatoid arthritis, retinitis, psoriasis and multiple sclerosis.

15. (canceled)

16. The pharmaceutical composition according to claim 6, for the treatment of cancer or an angiogenic disease.

17. The pharmaceutical composition according to claim 6, for the treatment of angiogenic diseases.

18-22. (canceled)

23. A method for the treatment or prevention of a disease, disorder or condition related to inhibition of VEGF binding to HS-GAGs, comprising administering to a subject in need thereof a therapeutically effective amount of a pharmaceutical composition comprising at least one compound of the general formula II in claim 6.

24. The method according to claim 23, wherein said disease, disorder or condition is an inflammatory or autoimmune disease, disorder or condition selected from the group consisting of sepsis, ischemia-reperfusion injury, cardiomyopathic disease, infectious meningitis, encephalitis, acute respiratory distress syndrome, organ/tissue transplant rejection (such as skin, kidney, heart, lung, liver, bone marrow, cornea, pancreas, small bowel), lupus, atherosclerosis, septic shock, post-ischemic leukocyte-mediated tissue damage, frost-bite injury or shock, acute leukocyte-mediated lung injury, acute pancreatitis, nephritis, asthma, traumatic shock, stroke, Crohn's disease, traumatic brain injury, acute and chronic inflammation, atopic dermatitis, uveitis, colitis, inflammatory bowel disease, rheumatoid arthritis, retinitis, psoriasis and multiple sclerosis.

25. (canceled)

26. The method according to claim 23, wherein said disease is cancer.
